(19) United States
(12) Patent Application Publication
(10) Pub. No.: US 2026/0331233 A1
(43) Pub. Date: Oct. 22, 2026

(54) BALANCING OF ENVIRONMENTAL ATTRIBUTES IN CHEMICAL PRODUCTION NETWORKS

(71) Applicant: BASF SE, Ludwigshafen am Rhein (DE)

(72) Inventors: Kurt WENZL, Ludwigshafen am Rhein (DE); Ana ALBA PEREZ, Ludwigshafen am Rhein (DE); Martin BINDER, Ludwigshafen am Rhein (DE); Christopher Alec ANDERLOHR, Ludwigshafen am Rhein (DE); Steffen KLOSTERHALFEN, Ludwigshafen am Rhein (DE); Christian KRUEGER, Ludwigshafen am Rhein (DE); Alessandro PISTILLO, Ludwigshafen am Rhein (DE); Bodo FREY, Ludwigshafen am Rhein (DE); Mathias FEYEN, Ludwigshafen am Rhein (DE); Claudius KORMANN, Ludwigshafen am Rhein (DE)

(21) Appl. No.: 19/110,322

(22) PCT Filed: May 4, 2023
(86) PCT No.: PCT/IB2023/054662

§ 371 Date: Mar. 10, 2025

Related U.S. Application Data
(60) Provisional application No. 63/416,091, filed on Oct. 14, 2022.

(30) Foreign Application Priority Data
Sep. 9, 2022 (EP) 22194793.0
Sep. 9, 2022 (EP) 22194800.3
Sep. 9, 2022 (EP) 22194808.6
Sep. 9, 2022 (EP) 22194815.1
Sep. 9, 2022 (EP) 22194818.5
Oct. 14, 2022 (EP) 22201672.7
Oct. 18, 2022 (EP) 22202183.4
Dec. 19, 2022 (EP) 111148811
Dec. 19, 2022 (WO) PCT/EP2022/086607
Dec. 19, 2022 (WO) PCT/EP2022/086610
Dec. 19, 2022 (WO) PCT/EP2022/086613
Dec. 19, 2022 (WO) PCT/EP2022/086614
Dec. 19, 2022 (WO) PCT/EP2022/086638
Dec. 19, 2022 (WO) PCT/EP2022/086646
Dec. 19, 2022 (WO) PCT/EP2022/086655
Dec. 19, 2022 (WO) PCT/EP2022/086671
Dec. 19, 2022 (WO) PCT/EP2022/086683
Dec. 19, 2022 (WO) PCT/EP2022/086684
Dec. 19, 2022 (WO) PCT/EP2022/086689
Dec. 19, 2022 (WO) PCT/EP2022/086696
Dec. 19, 2022 (WO) PCT/EP2022/086699
Dec. 19, 2022 (WO) PCT/EP2022/086701
Dec. 19, 2022 (WO) PCT/EP2022/086719
Dec. 19, 2022 (WO) PCT/EP2022/086744
Dec. 19, 2022 (WO) PCT/EP2022/086749
Dec. 19, 2022 (WO) PCT/EP2022/086762
Dec. 19, 2022 (WO) PCT/EP2022/086769
Dec. 19, 2022 (WO) PCT/EP2022/086772
Dec. 19, 2022 (WO) PCT/EP2022/086775
Dec. 19, 2022 (WO) PCT/EP2022/086810
Dec. 19, 2022 (WO) PCT/EP2022/086812
Apr. 27, 2023 (EP) 23170423.0

(51) Int. Cl. G16C 60/00; G05B 19/418

(52) U.S. Cl. CPC G16C 60/00; G05B 19/41865

(57) ABSTRACT

Disclosed are methods, apparatuses, and systems for producing at least one chemical product associated with one or more environmental attribute(s) and for assigning at least one environmental attribute to at least one chemical product produced by a chemical production network.

### TECHNICAL FIELD

The present disclosure relates to methods, apparatuses and systems for managing and attributing at least one environmental attribute associated with input material(s) to one or more chemical product.

### TECHNICAL BACKGROUND

In supply chains the environmental impact of each supply chain participant is of great interest. Transparency between the participants can aid collective reduction of environmental impacts to combat climate change. However, data sharing of environmental impact data is hindered by the lack of common data standards and the lack of trusted data platforms. In addition, the highly specific and centralized setup of data systems today makes exchange and sharing for collective action laborious. Hence, there is a need to develop metrices quantifying the environmental impact of produced products, to simplify data standards relating to environmental impact and to broadly enable a secure exchange of supply chain data relating to the environmental impact.

### SUMMARY OF THE INVENTION

In an aspect, the disclosure relates to a computer-implemented method for attributing at least one environmental attribute associated with an input material to one or more chemical products(s), wherein the one or more chemical products(s) are produced by a chemical production network using the input material(s), wherein the chemical production network chemically converts input materials via chemical intermediates to chemical products that exit the chemical production network, the method comprising:
providing input material data associated with the input material to an operating system of the chemical production network;
determining environmental attributes associated with the input material via a virtual production process;
allocating the environmental attributes associated with the input material to a virtual balancing account, wherein the virtual balancing account includes at least one attribution rule for attributing the environmental attributes associated with the input material to a chemical product;
providing a chemical product identifier associated with the chemical product and at least one target environmental attribute;
based on the chemical product identifier and the target environmental attribute, selecting at least one attribution rule;
determining via the at least one attribution rule at least one account for attributing one or more environmental attribute(s) from the at least one account to the chemical product identifier; and
assigning or attributing the one or more environmental attribute(s) to the chemical product identifier.

In another aspect, the disclosure relates to a computer-implemented method for monitoring environmental impact of one or more chemical product(s), wherein the one or more chemical products(s) are produced by a chemical production network using the input material(s), wherein the chemical production network chemically converts input materials via chemical intermediates to chemical products that exit the chemical production network, the method comprising:
providing input material data associated with the input material to an operating system of the chemical production network;
determining environmental attributes associated with the input material via a virtual production process;
allocating the environmental attributes associated with the input material to a virtual balancing account, wherein the virtual balancing account includes at least one attribution rule for attributing the environmental attributes associated with the input material to a chemical product;
providing a chemical product identifier associated with the chemical product and at least one target environmental attribute;
based on the chemical product identifier and the target environmental attribute, selecting at least one attribution rule;
determining via the at least one attribution rule at least one account for attributing one or more environmental attribute(s) from the at least one account to the chemical product identifier; and
assigning or attributing the one or more environmental attribute(s) to the chemical product identifier.

In another aspect, the disclosure relates to a computer-implemented method for monitoring environmental impact of one or more chemical product(s), wherein the one or more chemical products(s) are produced by a chemical production network using the input material(s), wherein the chemical production network chemically converts input materials via chemical intermediates to chemical products that exit the chemical production network, the method comprising:
providing multiple input material(s) associated with one or more environmental attribute(s) to the chemical production network, including a first input material and a second input material;
providing a first input material data associated with the first input material and a second input material data associated with the second input material;
providing at least one balancing account associated with the one or more environmental attribute(s) of the first input material and the second input material, wherein the virtual balancing account includes at least one attribution rule for attributing the environmental attributes associated with the input material to a chemical product;
providing a chemical product identifier associated with the chemical product and at least one target environmental attribute;
based on the chemical product identifier and the target environmental attribute, selecting at least one attribution rule;
determining via the at least one attribution rule at least one account for attributing one or more environmental attribute(s) from the at least one account to the chemical product identifier; and
assigning or attributing the one or more environmental attribute(s) to the chemical product identifier.

In another aspect, the disclosure relates to a computer-implemented method for attributing at least one environmental attribute associated with an input material to one or more chemical products(s), wherein the one or more chemical products(s) are produced by a chemical production network using the input material(s), wherein the chemical production network chemically converts input materials via chemical intermediates to chemical products that exit the chemical production network, the method comprising:
providing multiple input material(s) associated with one or more environmental attribute(s) to the chemical production network, including a first input material and a second input material;
providing a first input material data associated with the first input material and a second input material data associated with the second input material;
providing at least one balancing account associated with the one or more environmental attribute(s) of the first input material and the second input material, wherein the virtual balancing account includes at least one attribution rule for attributing the environmental attributes associated with the input material to a chemical product;
providing a chemical product identifier associated with the chemical product and at least one target environmental attribute;
based on the chemical product identifier and the target environmental attribute, selecting at least one attribution rule;
determining via the at least one attribution rule at least one account for attributing one or more environmental attribute(s) from the at least one account to the chemical product identifier; and
assigning or attributing the one or more environmental attribute(s) to the chemical product identifier.

In another aspect, the disclosure relates to a computer-implemented method for attributing at least one environmental attribute associated with an input material to one or more chemical products(s), the method comprising:
providing input material data associated with the input material to an operating system of the chemical production network;
producing environmental attributes associated with the input material via a virtual production process;
allocating the environmental attributes associated with the input material to a virtual balancing account, wherein the virtual balancing account includes at least one attribution rule for attributing the environmental attributes associated with the input material to a chemical product;
providing a chemical product identifier associated with the chemical product and at least one target environmental attribute;
selecting at least one attribution rule based, at least in part, on the chemical product identifier and the target environmental attribute;
determining via the at least one attribution rule at least one account for attributing one or more environmental attribute(s) from the at least one account to a digital asset the chemical product identifier;
generating a digital asset that includes the chemical product identifier and the one or environmental attributes; and
linking the digital asset to the chemical product.

In another aspect, the disclosure relates to a computer-implemented method for monitoring environmental impact of one or more chemical product(s), the method comprising:
providing input material data associated with the input material to an operating system of the chemical production network;
producing environmental attributes associated with the input material via a virtual production process;
allocating the environmental attributes associated with the input material to a virtual balancing account, wherein the virtual balancing account includes at least one attribution rule for attributing the environmental attributes associated with the input material to a chemical product;
providing a chemical product identifier associated with the chemical product and at least one target environmental attribute;
selecting at least one attribution rule based, at least in part, on the chemical product identifier and the target environmental attribute;
determining via the at least one attribution rule at least one account for attributing one or more environmental attribute(s) from the at least one account to a digital asset the chemical product identifier; and
generating a digital asset that includes the chemical product identifier and the one or environmental attributes; and
linking the digital asset to the chemical product.

In another aspect, the disclosure relates to an apparatus for assigning or attributing at least one environmental attribute associated with input material(s) to one or more chemical product(s), wherein the one or more chemical product(s)are produced by a chemical production network using at least one of the input material(s), the apparatus comprising:
a virtual production module configured to
   receive input material data associated with the at least one input material and to
   produce environmental attributes associated with the at least one input material;
a balancing module configured to provide at least one account for balancing the environmental attributes produced by the virtual production module;
an attribution module configured to provide at least one attribution rule for attributing environmental attributes associated with input material(s) to chemical product(s);
a digital asset provider configured to provide at least one chemical product identifier associated with the chemical product and at least one target environmental attribute for the chemical product; and
an outbound allocator configured to
   select based on the chemical product identifier and the target environmental attribute at least one attribution rule,
   determine at least one account for assigning or attributing one or more environmental attribute(s) from the account to the digital asset via the at least one attribution rule, and
   assigning or attributing the one or more environmental attribute(s) to the digital asset.

In another aspect, the disclosure relates to an apparatus for monitoring environmental impact of one or more chemical product(s), wherein the one or more chemical product(s)are produced by a chemical production network using at least one of the input material(s), the apparatus comprising:
a virtual production module configured to
   receive input material data associated with the at least one input material and to
   produce environmental attributes associated with the at least one input material;
a balancing module configured to provide at least one account for balancing the environmental attributes produced by the virtual production module;
an attribution module configured to provide at least one attribution rule for attributing environmental attributes associated with input material(s) to chemical product(s);
a digital asset provider configured to provide at least one chemical product identifier associated with the chemical product and at least one target environmental attribute for the chemical product; and
an outbound allocator configured to
   select based on the chemical product identifier and the target environmental attribute at least one attribution rule,
   determine at least one account for assigning or attributing one or more environmental attribute(s) from the account to the digital asset via the at least one attribution rule, and
   assigning or attributing the one or more environmental attribute(s) to the digital asset.

In another aspect, the disclosure relates to a digital operating system of a chemical production network, wherein the chemical production network chemically converts input materials via chemical intermediates to chemical products that exit the chemical production network, the digital operating system comprising:
a virtual production module configured to
   receive input material data associated with the at least one input material and to
   produce environmental attributes associated with the at least one input material;
a balancing module configured to provide at least one account for balancing the environmental attributes produced by the virtual production module;
a digital asset provider configured to provide at least one chemical product identifier associated with the chemical product and at least one target environmental attribute for the chemical product; and
an outbound allocator configured to
   select based on the at least one chemical product identifier and the at least one target environmental attribute at least one attribution rule,
   determine at least one account for assigning or attributing one or more environmental attribute(s) from the account to the digital asset via the at least one attribution rule, and
   assigning or attributing the one or more environmental attribute(s) to the digital asset.

In yet another aspect disclosed is a computer element, in particular a computer program product or a computer readable medium, with instructions, which when executed on one or more computing node(s) are configured to carry out the steps of any of the methods disclosed herein. In yet another aspect disclosed is a computer element, in particular a computer program product or a computer readable medium, with instructions, which when executed by a processor cause any of the apparatuses disclosed herein to perform any of the methods disclosed herein.

Disclosed is in yet another aspect the use of the chemical product associated with one or more environmental attribute(s) as provided by any of the methods disclosed herein and/or produced by a chemical production network as provided by any of the methods disclosed herein to produce at least one discrete product or at least one end product of a product supply chain associated with the one or more environmental attribute(s). Disclosed is in yet another aspect a method for producing at least one discrete product or at least one end product of a product supply chain associated with the one or more environmental attribute(s), wherein the chemical product associated with one or more environmental attribute(s) as provided by any of the methods disclosed herein and/or produced by a chemical production network as provided by any of the methods disclosed herein is provided and/or used to produce the at least one discrete product or at least one end product of a product supply chain associated with the one or more environmental attribute(s).

In yet another aspect the present disclosure relates to a computer element with instructions, which when executed on one or more computing node(s) is configured to carry out the steps of the method(s) of the present disclosure or configured to be carried out by the apparatus(es) of the present disclosure.

Any disclosure, embodiments and examples described herein relate to the methods, the systems, apparatuses, chemical products and computer elements lined out above and below.

Advantageously, the benefits provided by any of the embodiments and examples equally apply to all other embodiments and examples.

### EMBODIMENTS

The methods, apparatuses, systems, and computer elements disclosed herein provide an efficient way to track environmental attributes in chemical processing and provide chemical products with positive environmental impact through the value chain. By using a virtual accounting system with attribution rules for balancing environmental attributes associated with input materials, such attributes can be efficiently assigned to chemical products produced in chemical production networks. Specifically for chemical networks that produce more than one chemical product from more than one input material via interconnected, connected and non-connected production chains, the use of the virtual accounting system with attribution rules allows to reliably assign environmental attributes in line with the physical setup of the chemical production network and to tailor the digital assets associated with the chemical product to the needs of customers. The virtual accounting system and the associated metadata structure further allows to decouple the complexity in material flow of chemical production networks while still allowing to tailor environmental impact to each chemical product. This way the environmental impact of the produced chemical product can be determined in line with the physical set up of the chemical production network and the tailored needs of customers. Moreover, the environmental property of the chemical products produced by the chemical production network can be made transparent to customers further processing the chemical products. By providing chemical product identifiers associated with at least one environmental attribute, the environmental attributes and as such the digital asset attached to the chemical product can be adjusted to customer needs.

The methods, apparatuses, systems, and computer elements further disclosed herein provide an efficient way to track environmental attributes in chemical processing and to provide chemical products with positive environmental impact through the value chain. By using attribution rules environmental attributes associated with input materials can be efficiently attributed to environmental attributes associated with chemical products. Specifically for chemical networks that produce more than one chemical product from more than one input material via interconnected, connected and non-connected production chains, the use of attribution rules allows to reliably adjust the attribution mechanisms in line with the physical setup of the chemical production network. The use of virtual accounts for balancing environmental attributes further allows to abstract the complexity of chemical production networks while still allowing to assign environmental impact to chemical products. This way the environmental impact of the produced chemical product or chemical product can be determined in line with the physical set up of the chemical production network. Moreover, the environmental property of the chemical products produced in the chemical production network can be made transparent to customers further processing the chemical products. By providing a chemical product identifier associated with at least one target environmental attribute the environmental attributes may even be adjusted to customer needs.

The methods, apparatuses, systems, and computer elements further disclosed herein provide an efficient way to track environmental attributes in chemical processing and to provide chemical products with positive environmental impact through the value chain. By using a digital asset that is linked (or assigned, attributed, allocated, attached) to a chemical product, customers can easily request and select sustainable products (e.g., products with renewable, bio-based and/or a recycled content). They can use the digital asset to identify ways to make the value chain more sustainable. The digital asset also provides a way for chemical production networks to speed the transformation of the use of sustainable feedstocks as input materials and the production of chemical products at least partly based on sustainable materials. Specifically for chemical networks that produce more than one chemical product from more than one input material via interconnected, connected and non-connected production chains, the use of a digital asset enables the attribution of environmental attributes in line with the physical setup of the chemical production network. The use of digital assets enables the abstraction of the complexity of chemical production networks while still allowing to assign environmental impact to chemical products. This way the environmental impact of the produced chemical product(s) can be determined in line with the physical set up of the chemical production network. Moreover, the environmental property of the chemical products produced in the chemical production network can be made transparent to customers further processing the chemical products. By providing a digital asset with a chemical product identifier associated with at least one target environmental attribute the environmental attributes may even be adjusted to customer needs. Assigning or attributing at least one environmental attribute associated with input material(s) to chemical product(s) may include the linking of an input material identifier or a chemical product identifier with the environmental attribute. The input material identifier or the chemical product identifier may be associated with the physical entity of the input material or the chemical product, respectively. This way the virtual identifier of a material may be uniquely linked to the physical material. Such linking may include a physical or virtual link of identifiers uniquely associated with the physical material. For physical linking a tag or code may be physically connected to the material, e.g. by printing a QR code on the packaging. For virtual linking different identifiers associated with the physical material may be linked. For example, an order number, a batch number, LOT number or a combination thereof may be linked.

The environmental attribute may be a digital asset associated with the input material or chemical product. The environmental attribute may digitally specify the environmental impact of the input material or the chemical product. The environmental attribute may relate to a carbon footprint. The environmental attribute may relate to a renewable, a bio-based and/or a recycled content e.g., of the input material and/or chemical product. The environmental attribute may include a qualitative data point relating to the type of impact e.g., in view of the input material or the chemical product. The environmental attribute may specify a type such as recycled, renewable and/or bio-based. The qualitative data point may be converted to a quantitative measure such as balancing units. The environmental attribute may include a quantitate data point relating to the type of impact e.g., in view of the input material or the chemical product recycled content, renewable content or bio-based content. The environmental attribute may specify recycled, renewable and/or bio-based content. The term sustainable may refer to material that has renewable, bio-based and/or recycled content. For example, a sustainable input material may include recycled, renewable and/or bio-based content. Similarly, a sustainable chemical product may include recycled, renewable and/or bio-based content. The environmental attribute may include further environmental characteristics of the input(s) or the chemical product(s).

Environmental attribute(s) may refer to any property or characteristic related to the environmental impact. Such property may be a property or characteristic of an input material(s) and/or a chemical product(s). The environmental attribute may indicate an environmental performance of an input material(s), the chemical production network and/or chemical product(s). The environmental attribute may be derived from properties of the input material(s), the chemical production network and/or the chemical product(s). The environmental attribute may be associated with the environmental impact of one or more material(s) at any stage during their lifecycle. The stages of the material or product lifecycle may include the stages of providing raw material, producing products, such as intermediate products or end products, using products, treating end-of-life products, recycling end-of-life products, disposing end-of-life products, reusing components from end-of-life products or any subset of stages. The environmental attribute may be tracked through any activity of one or more entities participating at any stage of the lifecycle of one or more material(s) or product(s). Environmental attributes associated with any activity of one or more entities participating at any stage of the lifecycle of one or more material(s) or product(s) may be accumulated or aggregated.

The environmental attribute may include one or more characteristic(s) that are attributable to environmental or sustainability impact of the input material(s), chemical product(s), intermediate product(s) and/or end product(s). The environmental attribute may include environmental, technical, recyclability or circularity characteristics(s) associated with the environmental impact of the input material(s), chemical product(s), intermediate product(s) and/or end product(s).

Environmental characteristic(s) may specify or quantify ecological criteria associated with the environmental impact of an input material, intermediate product, and/or a chemical product. Environmental characteristic(s) may be or may be produced or derived from measurements taken during the lifecycle of input material(s), chemical product(s), intermediate product(s) and/or end product(s). Environmental characteristic(s) may for example include impact categories such as carbon footprint, greenhouse gas emissions or global warming potential, primary energy demand, cumulative energy demand, biotic and abiotic resource consumption, air emissions, stratospheric ozone depletion potential, ozone formation, terrestrial and/or marine acidification, water consumption, water depletion, water availability, water pollution, noise pollution, freshwater and/or marine eutrophication potential, human carcinogenic and/or non-carcinogenic toxicity, photochemical oxidant formation, particulate matter formation, terrestrial, freshwater and/or marine ecotoxicity, ionizing radiation, agricultural and/or urban land occupation, land transformation, land use, indirect land use, deforestation, biodiversity, mineral resource consumption, and/or fossil resource consumption. Environmental characteristic(s) may be calculated from combinations of one of more environmental characteristics. Environmental characteristic(s) may for example include material or product characteristics related to the production of the material or product like recycled content, bio-based content, renewable content, bio based, vegan, halal, kosher, palm oil-free, natural or the like. Technical characteristic(s) may specify or quantify material or product performance at least indirectly associated with the environmental impact. Technical characteristic(s) may for example include product composition data, bill of materials, product specification data, product component data, product safety data, application property data, application instructions or product quality data.

Technical characteristic(s) may be or may be produced from measurements taken during the lifecycle of one or more material(s) or product(s). Technical characteristics may be determined at any stage of the material or product lifecycle and may characterize the material or product performance for such stage or up to such stage. Technical characteristic(s) may for example include composition data, input in the production process, bill of materials, product or material specification data, product or material component data, product or material safety data, application property data, application instructions or product or material quality data. Technical characteristic(s) may for example include physical, chemical or further properties of the material or product.

Circularity characteristic(s) may specify or quantify the material or product life cycle characteristics associated with circular uses. Circularity characteristic(s) may be or may be produced from measurements taken during the lifecycle of one or more material(s) or product(s). Circularity characteristic(s) may be or may be produced from circular data recorded in one or more prior lifecycle(s) including reuse. Circularity characteristics may be determined at any stage of the material or product lifecycle and may characterize the reuse or recycling performance for such stage or up to such stage. Circularity characteristic(s) may relate to technical, mechanical, chemical and/or biological recycling. Circularity characteristic(s) may for example include recycling data, reuse rate, recycling rate, recycling loops, reuse reused product performance, reused material or product quality or the like. Further circularity material characteristics may be derived by combining circularity characteristic(s).

Recyclability characteristic(s) may specify or quantify the material or product life cycle characteristics associated with recycling uses. Recyclability characteristic(s) may include the composition of the material including specifically tailored constituents making the material suitable for recycling.

Recyclability characteristic(s) may be or may be produced from measurements taken during the lifecycle of one or more materials or product(s). Recyclability characteristic(s) may be or may be produced from recycling data recorded in one or more prior lifecycle(s). Recyclability characteristics may be determined at any stage of the material or product lifecycle and may characterize the recycling performance for such stage or up to such stage. Recyclability characteristic(s) may for example include recycling data, recyclability data, efficiency of recycling or the like.

The term “input material” as used in the present disclosure may refer to any good which is bought from suppliers and brought to the production plant. The input material may include starting material used in the production process of the production plant to produce the product. A input material can be on any step along the value chain like the product described above. This means, the product of the one production plant can be the input material of the other production plant. Input material can also include very fundamental goods like air, water, natural gas or salt. Input material(s) may refer to petrochemical feedstocks such as naphtha, crude oil, and natural gas, or intermediates from feedstocks that in turn require a certain amount of naphtha, crude oil, and natural gas.

Chemical products or input material(s) may include or be any material produced by the chemical production network using at least one input material. The chemical product may comprise or be any chemical product produced by the chemical production network and provided at any exit point of the chemical production network. The input material may comprise or be any input material entering the chemical production network and provided at any entry point of the chemical production network.

The chemical product or output material may be produced from input materials by the chemical production network. The chemical product or output material may comprise any material leaving the system boundary of the chemical production network. The input material may be any material entering the system boundary of the chemical production network. The chemical product may be an intermediate or end product produced from the target or output material.

The chemical production network may comprise one or more entry point(s) at which input materials are provided to the chemical production network. The chemical production network may comprise one or more exit point(s) at which chemical products are provided from the chemical production network. The chemical production network may include multiple chemical processes for producing one or more chemical product(s) from one or more input material(s). The chemical process may convert one or more input material(s) to one or more chemical product(s). The chemical process may chemically, physically, mechanically and/or thermally convert one or more input material(s) to one or more chemical product(s).

Calculated product characteristics may refer to Product Carbon Footprint (PCF), energy consumption, water consumption, crude oil consumption, labor (e.g., person hours associated with producing a product), social burdens (e.g., injuries and/or accidents associated with the production of a product), and other products characteristics that can be measured and calculated.

The material may comprise any chemical product obtained from chemical processes. The material may be obtained by mixing, separating, chemical reaction or natural reaction of chemical substances. The material may be a raw material fed to the chemical production network, an educt to a chemical process, an intermediate in a chemical process or a product of a chemical product. The material may be obtained from chemical reactions as well as natural chemical products. Natural chemical products may encompass any chemical substance that is naturally occurring, i.e. any unprocessed chemical substance that is found in nature, such as chemicals from plants, micro-organisms, animals, the earth and the sea or any chemical substance that is found in nature and extracted using a process that does not change its chemical composition. Natural chemical products may include biologicals like enzymes as well naturally occurring inorganic or organic chemical products. Natural chemical products can be isolated and purified prior to their use or they can be used in unisolated and/or unpurified form. Chemical products obtained from chemical reactions may be any inorganic or organic chemical product obtained by reacting inorganic and/or organic chemical reactants. The inorganic and organic chemical reactants may be naturally occurring chemical products or can be chemical products obtained from chemical reactions. Chemical reactions may include any chemical reaction commonly known in the state of the art in which the reactants are converted to one or more different chemical products. Chemical reactions may involve the use of catalysts, enzymes, bacteria, etc. to achieve the chemical reaction between the reactants.

Chemical production networks may include multiple types of production processes for producing different chemical products from input materials. The chemical production network may include a complex production network producing multiple chemical products in multiple production or value chains. A production or value chain may include one or more process(es) configured to produce one chemical product or chemical product class from one or more input material(s). The chemical production network may include connected, interconnected and/or non-connected production chains. The chemical production network may produce from input materials multiple intermediates and from intermediates one or more chemical products. Input material may enter the chemical production network at entry points. Chemical products may leave the production network at exit points (or feed-out points).

The chemical production network may comprise one or more entry points at which input materials are provided to the chemical production network. Input material may include fossil material, non-fossil material or both. Fossil input material may include crude oil, natural gas or coal. Non fossil input material may include renewable material, bio-based material or recycled materials. Input material may include feedstock for a gasification plant, a steam cracker or synthesis gas plant. Input material may include synthesis gas produced from fossil feedstock, non-fossil feedstock or both. Input material may include for example pyrolysis oil from recycled waste, syngas produced from recycled waste, naphtha produced from bio-based material (bio-naphtha), methane from bio-based material (bio-methane), biogas produced from the decomposition of organic materials or combinations thereof Input material may be provided to at least one gasification plant, steam cracker or synthesis gas plant, or any plant of the production chain for downstream products such as nitrogen, ammonia, methanol, ethylene, propylene, sulfur or the like.

The input material associated with one or more environmental attribute(s) provided to the entry point of the chemical production network may include recycled input materials including, but not limited to, recycled pyrolysis oil, recycled pyrolysis gas, recycled synthesis gas, recycled hydrogen, recycled naphtha, recycled methane, recycled ethane, recycled propane, recycled chemicals or combinations thereof. Recycled chemicals may include, but may not be limited to, recycled ammonia, recycled methanol, recycled ethylene, recycled propylene, recycled benzene, recycled toluene, recycled xylene or combinations thereof. In the context provided here recycled input material may include any material that at least in part includes recycled content and/or is at least in part produced from recycled content. The recycled content may be physically and/or chemically traceable.

The input material associated with one or more environmental attribute(s) provided to the entry point of the chemical production network may include renewable input materials including, but not limited to, renewable pyrolysis oil, renewable pyrolysis gas, renewable synthesis gas, renewable hydrogen, renewable naphtha, renewable methane, renewable ethane, renewable propane, renewable chemicals or combinations thereof. Renewable chemicals may include, but may not be limited to, renewable ammonia, renewable methanol, renewable ethylene, renewable propylene, renewable benzene, renewable toluene, renewable xylene or combinations thereof. In the context provided here renewable input material may include any material that at least in part includes renewable content and/or is at least in part produced from renewable content. The renewable content may be physically and/or chemically traceable.

The input material associated with one or more environmental attribute(s) provided to the entry point of the chemical production network may include bio-based input materials including, but not limited to, bio-based pyrolysis oil, bio-based pyrolysis gas, bio-based synthesis gas, bio-based hydrogen, bio-based naphtha, bio-based methane, bio-based ethane, bio-based propane, bio-based chemicals or combinations thereof. Bio-based chemicals may include, but may not be limited to, bio-based ammonia, bio-based methanol, bio-based ethylene, bio-based propylene, bio-based benzene, bio-based toluene, bio-based xylene or combinations thereof. In the context provided here bio-based input material may include any material that at least in part includes bio-based content and/or is at least in part produced from bio-based content. The bio-based content may be physically and/or chemically traceable.

The chemical production network may include identity preserving or segregated production chains. Identity preserving or segregated in this context may refer to the environmental attributes of the input materials being preserved or segregated in the production chains. Examples are bio-based-renewable or recycled input materials used to produce the chemical product without fossil content. Further examples are fossil input materials used to produce the chemical products with fossil content. Chemical production networks may include non-identity preserving or non-segregated production chains. Non-identity preserving or non-segregated in this context may refer to input materials associated with environmental attributes being mixed. For example, non-identity preserving or non-segregated in this context refers to input materials associated with environmental attributes being mixed with fossil input materials in the production chains. Examples are fossil and renewable input materials mixed to produce the chemical product with fossil and renewable content.

The chemical production network may include multiple production steps per production chain. The production steps included in the chemical production network may be defined by the physical system boundary of the chemical production network. The system boundary may be defined by location or control over production processes. The system boundary may be defined by the site of the chemical production network. The system boundary may be defined by production processes controlled by one entity or multiple entities jointly. The system boundary may be defined by production or value chain with staggered production processes to an end product, which may be controlled by multiple entities separately. For example, the chemical production network may include a waste collection step, a waste sorting step, a recycling step such as chemical recycling through pyrolysis, a cracking step such as steam cracking, a separation step to separate outputs of one process step and further processing steps to convert such outputs to chemical products leaving the system boundary of the chemical production network. The entry points (or feed-in points) of the chemical production network may be marked by the entry of input materials to the chemical production network. The input materials entering the chemical production network may be used to produce one or more chemical products. The chemical products may leave the physical system boundary of the chemical production network. The exit points of the chemical production network may be marked by the exit of chemical products from the chemical production network.

The production operating apparatus may be configured to provide a decentral identifier associated with a physical entity of a produced chemical product. The production operating apparatus may be configured to link the decentral identifier to a physical identifier of the chemical product. The production operating apparatus may be configured to assign the decentral identifier to the physical identifier connected to the chemical product. The production operating apparatus may be configured to assign the decentral identifier to the physical identifier physically connected to the chemical product.

The decentral identifier may relate to data associated with at least one chemical product produced by the chemical production network, wherein the one or more environmental attribute(s) associated with the at least one chemical product are derived from one or more environmental attribute(s) associated with the input material(s). The one or more environmental attribute(s) associated with the chemical product(s) may be associated with the one or more input material(s) and/or the chemical process(s) used to produce the chemical product(s). The decentral identifier may relate to any identifier uniquely associated with the chemical product. The decentral identifier may be associated with the physical entity of the chemical product. The decentral identifier may refer to a single batch of chemical product. The decentral identifier may be associated with a group of chemical product(s). The identifier may refer to multiple physical entities of the chemical product(s). The decentral identifier may be associated with continuous or semi-continuous stream of the chemical product. The identifier may refer to a stream of the chemical product e.g. over a certain time period.

The decentral identifier may comprise any unique identifier uniquely associated with the chemical product(s) such as the environmental attributes. The decentral identifier may include a Universally Unique IDentifier (UUID) or a Digital IDentifier (DID). The decentral identifier may be issued by a central or decentral identity issuer. The decentral identifier may be linked to authentication and/or authorization information. Via the decentral identifier and its unique association with the output material producer and output material data, such as the environmental attributes, access to the output material data may be controlled by the output material producer. This contrasts with central authority schemes, where identifiers are provided by such central authority and access to data is controlled by such central authority. Decentral in this context refers to the usage of the identifier in implementation as controlled by the data owner, such as the output material producer.

The virtual balancing account (or digital inventory) may refer to a digital storage structure that stores data related to environmental attributes. The account may be associated with metadata identifying the account for balancing environmental attributes. The account may be associated with metadata identifying the environmental attributes and the environmental or balancing units allocated to the account. The account may be associated with metadata identifying the production chain the account is associated with. The account may be associated with metadata identifying the input or chemical product the account is associated with. The account may be part of a balancing system including multiple accounts. The account may hold environmental attributes for transaction. Environmental attributes may be allocated, added, deleted, withdrawn, or deducted from the account. The virtual balancing account may be associated with environmental attribute types such as recycled or renewable. The virtual balancing account may by associated with input material types such as pyrolysis oil, bio-naphtha, bio-methane, bio-gas or combinations thereof. The virtual balancing account associated with the environmental attribute type recycled may be further associated with waste-stream type such as mixed plastics waste, specific end product waste, e.g., tiers waste or foam waste, post-consumer waste, pre-consumer waste or combinations thereof. The virtual balancing account may be associated with an allocation scheme such as segregated allocation, non-segregated allocation like book and claim, mass balance with free attribution, mass balance without free attribution or combinations thereof.

The at least one attribution rule may specify the attribution scheme associated with the account for balancing environmental attributes. The at least one attribution rule may specify the attribution of environmental attributes associated with input materials and the chemical production network to environmental attributes associated with chemical products. The at least one attribution rule may depend on a chemical product identifier and an environmental attribute. The at least one attribution rule may include instructions for attributing environmental attributes from input materials to at least one account for balancing environmental attributes. The at least one attribution rule may include instructions for deducting environmental attributes from at least one account for balancing environmental attributes. The at least one attribution rule may include instructions for attributing environmental attributes from the account to chemical products or chemical product identifiers.

The at least one attribution rule may be associated with environmental attribute types that relate to certified or non-certified environmental attributes. The at least one attribution rule may be associated with environmental attribute types that relate to input material dependent environmental attributes. The at least one attribution rule may be associated with environmental attribute types that relate to chemical network or production chain dependent environmental attributes. The at least one attribution rule may be associated with environmental attribute types that relate to chemical product dependent environmental attributes. The at least one attribution rule may be associated with environmental attribute types that relate to certified or non-certified environmental attributes. The at least one attribution rule may be associated with environmental attribute types that relate to environmental attributes certified under specific certification schemes. The at least one attribution rule may be associated with environmental attribute types that relate to environmental attributes adhering to specific attribution schemes.

The at least one attribution rule may be associated with at least one chemical production network producing the at least one chemical product(s). The at least one attribution rule may be associated with at least one chemical production network including one or more production chains. The at least one attribution rule may be associated with at least one chemical production network including one or more process steps converting input material(s) to one or more intermediate(s) and/or one or more chemical product(s). The at least one attribution rule may be associated with at least one process setup of the chemical production network.

The at least one attribution rule may be associated with at least one attribution scheme specifying the balancing or environmental attributes. The at least one attribution rule may be associated with at least one segregated or non-segregated attribution scheme. The at least one attribution rule may be associated with at least one non-segregated attribution scheme. The at least one attribution rule may be associated with one or more non-segregated attribution schemes, such as a mass balance scheme with free attribution, a mass balance scheme without free attribution or a book-and-claim scheme.

The at least one attribution rule may be associated with at least one input material characterized by at least one environmental attribute type. The at least one attribution rule may be associated with at least one input material entering the chemical production network. The at least one attribution rule may be associated with at least one input material used to produce one or more chemical product(s).

The at least one attribution rule may be associated with at least one chemical product characterized by at least one environmental attribute type. The at least one attribution rule may be associated with at least one chemical product type exiting the chemical production network. The at least one attribution rule may be associated with at least one chemical product type produced from one or more input material(s).

The account for balancing environmental attributes and the at least one attribution rule may be associated at least in part with corresponding metadata. The account for balancing environmental attributes and the at least one attribution rule may be associated with corresponding metadata. The account for balancing environmental attributes and the at least one attribution rule may be associated with partially corresponding metadata. The set of metadata associated with the account for balancing environmental attributes and the at least one attribution rule may match in all data points of the meta data. The set of metadata associated with the account for balancing environmental attributes and the at least one attribution rule may relate to at least one environmental attribute type, at least one chemical production network, at least one production chain, at least one attribution scheme, at least one input material type, at least one chemical product type or combinations thereof. The input material type may relate to characteristics of input material such as recycled material, biobased material or renewable material. The input material type may relate to the material and its use or entry points such as pyrolysis oil for input to a steam cracker or syngas plant, bio-gas for input to a steam cracker or syngas plant. The input material type may relate to the geographic origin of the input material. The input material type may relate to the production process of the input material, such as mechanically or chemically recycled material.

The attribution rule may include instructions to determine one or more account(s) accessible for the at least one chemical product. The at least one attribution rule may include instructions to determine one or more account(s) accessible for the at least one chemical product and/or the environmental units accessible for the at least one chemical product. The attribution rule may be associated with metadata signifying the one or more account(s) accessible for the at least one chemical product. The attribution rule may include instructions to determine the one or more account(s) accessible for the at least one chemical product. The attribution rule may include instructions to verify or validate the one or more account(s) accessible for the at least one chemical product. The attribution rule may include instructions to determine, verify and/or validate the one or more account(s) accessible for the at least one chemical product. For determination of the one or more account(s) accessible for the at least one chemical product, the attribution rule may be associated with the chemical product.

The attribution rule may include instructions to determine the input material(s) used to produce the chemical product. The attribution rule may include instructions to access a bill of material comprising input material data, chemical product data and process data. From the bill of material, the environmental attribute types accessible for the at least one chemical product may be determined. From the environmental attribute types, the one or more account(s) accessible for the at least one chemical product may be determined.

The attribution rule may include instructions to match the metadata of the accounts with the chemical product type corresponding to the chemical product. For determination of the one or more account(s) accessible for the at least one chemical product, the attribution rule may be associated with the production chain. The attribution rule may include instructions to match the metadata of the accounts with the production chain. Such metadata matching may be executed for any combination of metadata associated to the account(s) and attribution rules as lined out above.

For verification of the one or more account(s) accessible for the at least one chemical product, the attribution rule may be associated with the chemical product type and one or more account(s). On verification the one or more account(s) accessible for the at least one chemical product may be determined and compared to the one or more account(s) associated with the attribution rule.

For validation of the one or more account(s) accessible for the at least one chemical product, the attribution rule may be associated with the chemical product type and one or more account(s). On validation the metadata and/or the balance of the one or more account(s) accessible for the at least one chemical product may be checked to be validly accessible.

The chemical product may be produced by the chemical production network to which the input material(s) associated with one or more environmental attribute(s) were provided. The chemical product may be produced by a production chain of the chemical production network to which the input material(s) associated with one or more environmental attribute(s) were provided. The chemical product may be produced from the input material(s) associated with one or more environmental attribute(s).

The identifier associated with the chemical product may include one or more identifier(s) relating to the chemical product. The identifier may relate to a chemical product class, a specific chemical product and/or properties of the chemical product such as environmental properties. The identifier may include a unique number uniquely associated with the chemical product class, the specific chemical product and/or the properties of the chemical product. The identifier may include one or more specific identifier(s), such as chemical product class identifier, specific chemical product identifier and/or property of the chemical product identifier. Such specific identifier(s) may be uniquely linked to the chemical product. For example, one or more property identifier(s) may be uniquely linked to the chemical product identifier. The chemical product identifier may be uniquely linked to the specific chemical product. This way the chemical product can be uniquely linked to a digital twin of the chemical product specifying specific properties of the chemical product.

The identifier associated with the chemical product may include one or more identifier(s) relating to one or more environmental attribute(s). The identifier may include an environmental attribute identifier, such as a unique environmental attribute identifier, relating to environmental attribute(s) assignable to chemical products. The environmental attribute identifier may relate to the chemical product class or the specific chemical product. For example, the environmental attribute identifier may relate to recycled content, bio-based content and/or renewable content as environmental attribute, each having their own unique each having their own unique material identifier. The specific environmental attribute or a specific combination of environmental attributes may be related to the unique environmental attribute identifier.

The identifier may include, be linked to or be related to a batch and/or order number, such as an unique batch and/or order number. The batch number may be linked to the physical entity of produced chemical product batches. The order number may be linked to the transaction specifying the shipment of the chemical product batch from the producer of the chemical product to the user further processing the chemical product.

In another embodiment, the virtual production process for environmental attributes associated with the input material further comprises determining the amount of the input material. The determination may be based on a bill of materials, a sales receipt, a recipe and/or any of a wide range of digital documents (e.g., input material data) associated with receipt of input material(s). An operating system may parse the input material data to determine the amount of input material that was received. The “amount” of the input material may refer to the volume, amount of substance, and/or mass of the input material.

In another embodiment, the virtual production process for environmental attributes associated with the input material further comprises determining a value associated with the input material. For example, an operating system may compute the difference in cost between a sustainable input material and the corresponding equivalent fossil input material to determine the value associated with the input material. The value may be based on average price, actual price, market price or other suitable values to determine the cost of the equivalent amount of fossil input materials. The operating system may store and track the amounts and values corresponding to sustainable input materials. For example, the values may be stored in digital inventories associated with environmental attributes or balancing units.

In another embodiment, an operating system may assign or attribute the one or more environmental attribute(s) to the chemical product identifier. This may include producing a digital asset that specifies a chemical product with the combination of the chemical product identifier and the one or more environmental attributes and assigning the digital asset to the chemical product. In another embodiment, the digital asset may uniquely specify the chemical product with the combination of the chemical product identifier and the one or more environmental attributes. In another embodiment, the chemical product identifier is associated with a product specification for the chemical product.

In another embodiment, the digital asset includes a value associated with the input material, wherein the value associated with the input material is related to a difference in cost between the input material and a corresponding amount of fossil input material. The value may be related to a difference in cost between the input material and the corresponding amount of fossil input material. The value may be based on average price, actual price, market price or other suitable values to determine the cost of the equivalent amount of fossil input materials.

In another embodiment, providing input material data associated with the input material may include providing a first input material data associated with the first input material and providing a second input material data associated with the second input material. In yet another embodiment, the method further comprises providing at least one balancing account associated with one or more environmental attribute(s) of the first input material and the second input material. In another embodiment, assigning or attributing the one or more environmental attribute(s) to the chemical product identifier may include generating a digital asset that includes the chemical product identifier and the one or environmental attribute(s) of at least one of the first input material and the second input material and linking the digital asset to the chemical product.

In another embodiment, the virtual production module is configured to receive input material data associated with the at least one input material, produce environmental attributes associated with the at least one input material, and determine an amount of the input material and determine a value associated with the input material.

In another embodiment, the environmental attribute relates to or is associated with a renewable, a bio-based and/or a recycled content. For example, input material from organic waste may be associated with the environmental attributes recycled and bio-based. Input material from organic waste may be bio-based and recycled input material. Further for example, input material from wooden waste may be associated with the environmental attributes recycled, bio-based and renewable. Input material from wooden waste may be bio-based, renewable and recycled input material. In the context provided here renewable, a bio-based and/or a recycled input material may include any material that at least in part includes renewable, a bio-based and/or a recycled content and/or is at least in part produced from renewable, a bio-based and/or a recycled content. The renewable, a bio-based and/or a recycled content may be physically and/or chemically traceable.

In another embodiment the virtual balancing system or the balancing account relates to or is associated with one or more characteristic(s) of the input material associated with one or more environmental attribute(s). The virtual balancing system or the balancing account may relate to one or more characteristic(s) of the input material that are attributable to environmental impact of the input material and the chemical product produced by the chemical production network. The characteristic(s) of the input material may include, and may not be limited to, an input material type, a waste stream type, a biomass type, a renewable type, an allocation scheme, or combinations thereof.

By specifying the characteristics of the input material, the environmental attribute and characteristics of input materials used to produce the chemical product may be tracked with more granularity. This enables a system to track the environmental attributes and characteristics of the input material more granularly. More granular tracking via the virtual accounting system in return enables assignment of environmental attributes from balancing accounts that are tailored to customer needs.

In another embodiment the virtual balancing system or the balancing account relates to or is associated with an input material type, a waste stream type, a biomass type, a renewable type, an allocation scheme, or combinations thereof. The virtual balancing system or the balancing account may be associated with metadata specifying the input material associated with one or more environmental attribute(s). The metadata may specify the input material associated with one or more environmental attribute(s) provided to the entry point of the chemical production network. The input material type may include, and may not be limited to, pyrolysis oil, pyrolysis gas, synthesis gas, hydrogen, naphtha, methane, ethane, propane, chemicals, or combinations thereof. Chemicals may include, but may not be limited to, ammonia, methanol, ethylene, propylene, benzene, toluene, xylene, or combinations thereof.

The virtual balancing system or the balancing account may be associated with metadata specifying an input material type, a waste stream type, a biomass type, a renewable type, an allocation scheme, or combinations thereof. As one example, recycled pyrolysis oil produced from plastics waste may be provided to the chemical production network as input material. The metadata may specify the input material type pyrolysis oil, the environmental attribute type recycled, the waste stream type mixed plastics waste, specific end product waste, post-consumer waste or pre-consumer waste, and/or the allocation scheme non-segregated scheme such as mass balance. As another example, bio-based naphtha may be provided to the chemical production network as input material. The metadata may specify the input material type naphtha, the environmental attribute type bio-based, the biomass type palm oil and/or the allocation scheme non-segregated scheme such as mass balance. As another example, bio-based methane may be provided to the chemical production network as input material. The metadata may specify the input material type methane, the environmental attribute type bio-based, the biomass type waste from agriculture and/or the allocation scheme non-segregated scheme such as mass balance.

In another embodiment the virtual balancing system or the balancing account is associated with input material types including pyrolysis oil, pyrolysis gas, synthesis gas, hydrogen, r-chemicals, bio-naphtha or bio-methane, wherein the balancing account is associated with pyrolysis or gasification and the waste stream the pyrolysis oil, pyrolysis gas, synthesis gas, hydrogen or r-chemicals is produced from, wherein the balancing account is associated with bio-naphtha or bio-methane and the renewable stream the bio-naphtha or bio-methane is produced from.

In another embodiment the virtual balancing system or the balancing account relates to or is associated with waste stream types. The waste stream types may be linked to at least the environmental attribute recycled. The waste stream types may relate to one or more waste materials or waste categories. Waste categories may include, and are not limited to, non-synthetic waste such as animal waste, vegetable or wooden waste, or synthetic waste, such as textile waste, paper waste, plastics waste, or rubber waste. The waste stream types may relate to the origin of the waste, such as the end-of-life product, the producer of the end-of-life product, the geolocation of the end-of-life product, the habitat of the end-of-life product, the consumer of the end-of-life product, or combinations thereof and may not be limited thereto. For example, in the case of recycled as environmental attribute and pyrolysis oil as input material type the waste type may specify plastics waste from tires. mixed plastics waste from packaging or mixed plastics waste from ocean cleanup.

In another embodiment the virtual balancing system or the balancing account relates to or is associated with biomass types or renewable types. The biomass type may be linked to at least the environmental attribute bio-based or renewable. The biomass type may relate to one or more raw materials the input material is produced from. Raw materials may include, and are not limited to, agricultural waste, manure, municipal waste, plant material, sewage, green waste, food waste, natural occurring fats, natural occurring oils, mixtures of natural occurring fats and oils, cooking oil, or animal fats. The biomass type may relate to the origin of the biomass, such as the producer of the biomass, the geolocation of the biomass, the habitat of the biomass, the consumer of the biomass, or combinations thereof and may not be limited thereto. For example, in the case of bio-based as environmental attribute and bio-naphtha as input material type the biomass type may specify oils and fats from plants (vegan). Further for example, in the case of bio-based as environmental attribute and biogas as input material type the biomass type may specify municipal waste.

In another embodiment the virtual balancing system or the balancing account is associated with metadata specifying the relationship between the input material(s) associated with one or more environmental attribute(s) and the chemical product. In another embodiment the relationship relates to the chemical production network, one or more production chain(s), one or more chemical product(s), one or more chemical product classes or combinations thereof.

The relationship may relate to the chemical production network. For example, the input material(s) associated with one or more environmental attribute(s) may be provided to the chemical production network producing the chemical product. The relationship may relate to one or more production chain(s) of the chemical production network producing the chemical product. For example, the input material(s) associated with one or more environmental attribute(s) may be provided to one or more production chain(s) of the chemical production network producing the chemical product. The relationship may relate to one or more chemical product class(es) produced by the chemical production network. For example, the input material(s) associated with one or more environmental attribute(s) may be provided to one or more production chain(s) of the chemical production network producing the chemical product class(es). The relationship may relate to one or more chemical product(s) produced by the chemical production network. For example, the input material(s) associated with one or more environmental attribute(s) may be provided to one or more production chain(s) of the chemical production network producing the one or more chemical product. The relationship may relate to the chemical production network, one or more production chain(s), one or more chemical product(s), one or more chemical product classes or combinations thereof. By tagging with the relationship metadata the input materials may be physically and/or chemically traceable. The metadata may be derived from a digital twin of the chemical production network (or a portion of the chemical production network). The metadata may be derived from a digital twin of the chemical production network (or a portion thereof) and a production schedule.

In another embodiment at least one target environmental attribute for the chemical product is provided and based on the at least one target environmental attribute at least one balancing account for deallocation the respective environmental attribute is selected. The steps may be performed at any stage of the method. For example, assigning the at least one environmental attribute to the at least one chemical product may include providing at least one target environmental attribute for the chemical product and based on the at least one target environmental attribute selecting at least one balancing account for deallocation of the respective environmental attribute. For example, at least one target environmental attribute for the chemical product may be provided and assigning the at least one environmental attribute to the at least one chemical product may include based on the at least one target environmental attribute selecting at least one balancing account for deallocation the respective environmental attribute.

Owing to the virtual accounting system and associated metadata granular target environmental attributes can be achieved. The target environmental attribute may relate to or be associated with a renewable, a bio-based and/or a recycled content. The target environmental attribute may further be related to or associated with metadata specifying an input material type, a waste stream type, a biomass type, a renewable type, an allocation scheme, or combinations thereof. Based on such metadata the metadata associated with or related to the virtual accounting system or the balancing accounts may be matched, and matching balancing accounts may be selected. The respective environmental attributes may be deallocated from the selected balancing accounts. The deallocated environmental attributes may be digitally assigned to the chemical product, e.g., by assigning the environmental attribute from the balancing account to the chemical product identifier.

In another embodiment assigning the at least one environmental attribute to the at least one chemical product includes providing at least one target environmental attribute for the chemical product,
checking the environmental attribute balance of the balancing account associated with the at least one target environmental attribute and assigning the at least one environmental attribute from the respective balancing account to the at least one chemical product, if the balance is sufficient, and/or
checking the respective balancing accounts are associated with input material(s) used to produce the chemical product and assigning the at least one environmental attribute from the respective balancing account to the at least one chemical product if the respective balancing account is associated with input material(s) used in the production chain of the chemical product. Input material(s) used to produce the chemical product may include input material(s) associated with environmental attributes at the entry to the chemical production network. The respective environmental attributes may be decoupled from the material flow by allocation to the balancing account. Input material(s) used to produce the chemical product may include input material(s) associated with environmental attribute(s) and input material(s) not associated with environmental attribute(s). The environmental attribute(s) may be decoupled from the material flow through the chemical production network by allocation to the balancing account.

Input material(s) used to produce the chemical product may include input material(s) provided at entry points to the chemical production network. The entry points to the chemical production network may signify any point of input material(s) entering the physical boundary of the chemical production network. Upon entry material data related to the one or more input material(s) and respective environmental attributes may be provided to a computing interface configured to allocate the environmental attribute(s) to the balancing account. The material data may include at least one material identifier and at least one environmental attribute. The material data may further include an amount of input material provided. The at least one environmental attribute may be allocated to the balancing account. This way the material flow of the input material(s) through the chemical production network may be decoupled from the environmental attributes and the environmental attributes may be allocated to chemical products fully or at least partially independent of material flows.

In another embodiment assigning at least one environmental attribute to the at least one chemical product includes providing a chemical product identifier associated with the produced chemical product and assigning the environmental attribute from the balancing account to the chemical product identifier. Assigning at least one environmental attribute associated with input material(s) to chemical product(s) may include the linking of the chemical product identifier with the environmental attribute. The chemical product identifier may be associated with the physical entity of the chemical product. This way the virtual identifier of a material may be uniquely linked to the physical material. Such linking may include a physical or virtual link of identifiers uniquely associated with the physical material. For physical linking a tag or code may be physically connected to the material, e.g., by printing a QR code on the packaging. For virtual linking different identifiers associated with the physical material may be linked. For example, an order number, a batch number, LOT number or a combination thereof may be linked.

### BRIEF DESCRIPTION OF THE DRAWINGS

In the following, the present disclosure is further described with reference to the enclosed figures. The same reference numbers in the drawings and this disclosure are intended to refer to the same or like elements, components, and/or parts.

FIG. 1 illustrates an example of a chemical production network producing one or more chemical product(s) from one or more input material(s) in connection with an operating system including an attribute management system.

FIG. 2 illustrates an example of a chemical production network producing one or more chemical product(s) from one or more input material(s) in connection with an operating system including an attribute management system for two or more environmental attributes.

FIG. 3 illustrates a virtual production system for producing sustainable chemical products by decoupling the environmental attributes of incoming sustainable inputs and producing balancing units.

FIG. 4 illustrates an amalgamating system for producing sustainable chemical products by combining environmental attributes with conventional products.

FIGS. 5a-c illustrate a part of a chemical production network producing multiple chemical product(s) from fossil and non-fossil input material(s).

FIGS. 6a-c illustrate examples of allocation schemes allocating the use of renewable or bio-based input materials to chemical products of the chemical production network.

FIG. 7 illustrates an example of a chemical production network with different allocation schemes.

FIG. 8a, b illustrate inbound and outbound allocation of environmental attributes for a chemical production network.

FIG. 9 illustrates an example of a method for assigning at least one environmental attribute to at least one chemical product produced by a chemical production network.

FIG. 10 illustrates an example of an apparatus for producing at least one chemical product associated with one or more environmental attribute(s) including an example method for converting and allocating to the virtual balancing account on inbound.

FIG. 11 illustrates an example of an apparatus for producing at least one chemical product associated with one or more environmental attribute(s) including an example method for converting and assigning to the chemical product on outbound.

FIG. 12 illustrates an example of chemical products produced by the chemical production network.

FIG. 13 illustrates an example of the converting to balancing units and assignment to chemical products.

FIG. 14a, b illustrate examples of data structures for assigning environmental attributes from the balancing account to the chemical product identifier.

FIGS. 15a-c illustrate examples of virtual balancing accounts and associated metadata.

FIG. 16 illustrates an example of a method for attributing or allocating at least one environmental attribute to a balancing account based on an attribution rule.

FIG. 17 illustrates examples of attribution rules for attributing or allocating at least one environmental attribute to a balancing account.

FIG. 18 illustrates an example of a method for assigning or attributing at least one environmental attribute to a chemical product id based on an attribution rule.

FIG. 19 illustrates examples of attribution rules for assigning or attributing at least one environmental attribute to a chemical product id based on an attribution rule.

FIG. 20 illustrates examples of attribution rule instructions for selecting at least one account.

FIG. 21 illustrates examples of attribution rule instructions for compatibility check of accounts.

FIG. 22 illustrates schematically another example of a method or apparatus for providing environmental attributes associated with output materials to a material user as data consumer via a decentral network.

FIG. 23 illustrates schematically an example of a method or apparatus for providing environmental attributes of output materials across value chains via the decentral network.

FIG. 24 illustrates an example of a method for producing at least one chemical product associated with at least one environmental attribute for a supply chain.

### DETAILED DESCRIPTION

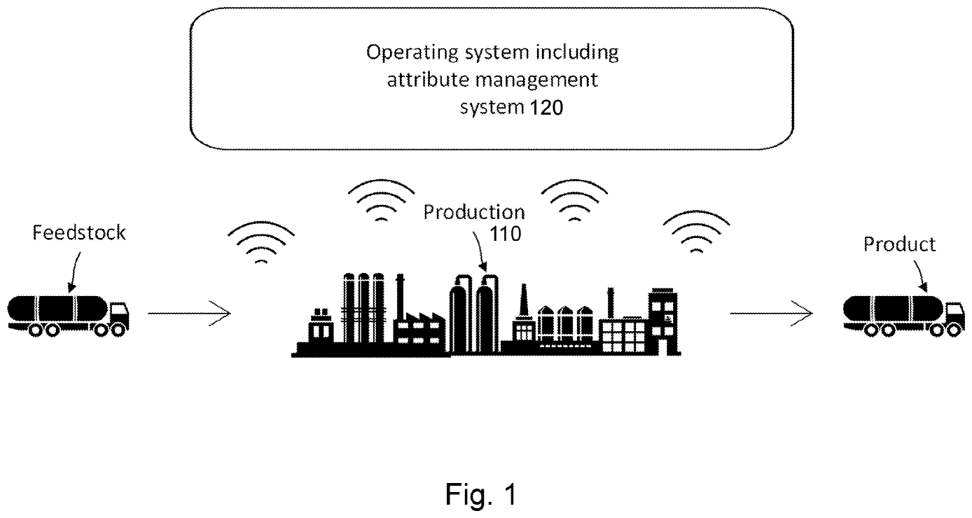

FIG. 1 illustrates an example of a chemical production network 110 producing one or more chemical product(s) from one or more input material(s) in connection with an operating system including an attribute management system 120. For producing one or more chemical product(s) different input materials (feedstocks) may be provided as physical inputs from material providers or suppliers. The chemical products produced from the input materials may have one or more properties related to the environmental impact of the input materials or the chemical products produced from the input materials, that may be signified by the environmental attributes.

The chemical production network may include multiple interlinked processing steps. The chemical production network may be an integrated chemical production network with connected or interconnected production chains. The chemical production network may include multiple different production chains that have at least one intermediate product in common. The chemical production network may include multiple stages of the chemical value chain. The chemical production network may include the producing, refining, processing and/or purification of gas or crude oil. The chemical production network may include a stream cracker, or a syngas plant connected to multiple production chains that output chemical products from the effluent of the steam cracker or syngas plants. The chemical production network may include multiple production chains that produce from one or more input material(s) chemical products that exit the chemical production network. The chemical production network may include multiple tiers of a chemical value chain. The chemical production network may include physically connected or interconnected supply chains and/or production sites. The production sites may be at the same location or at different locations. In the latter case, the production sites may be connected or interconnected by means of dedicated transportation systems such as pipelines, supply chain vehicles, like trucks, ships or other cargo transportation means.

The chemical production network may chemically convert input materials via chemical intermediates to one or more chemical product(s) that exit the chemical production network. The chemical production network may convert input material(s) by way of chemical conversion to one or more chemical product(s).

The input material(s) may be fed into the chemical production network at any entry point. The input material(s) may be fed into the chemical production network at the start of the chemical production network. Input materials may for example make up the feedstock of a steam cracker. The input material may include a bio-based, a recycled and/or a fossil input material for the manufacture of chemical intermediates and chemical products.

The chemical production network may include multiple production steps. The production steps included in the chemical network may be defined by the system boundary of the chemical production network. The system boundary may be defined by location or control over production processes. The system boundary may be defined by the site of the chemical production network. The system boundary may be defined by production processes controlled by one entity or multiple entities jointly. The system boundary may be defined by a value chain with staggered production processes to an end product, which may be controlled by multiple entities separately. The chemical production network may include a waste collection and sorting step, a recycling step such as pyrolysis, a cracking step such as steam cracking, a separation step to separate outputs of one process step and further processing steps to convert such outputs to a chemical product leaving the system boundary of the chemical production network.

The operating system 120 of the chemical production network may monitor and/or control the chemical production network based on operating parameters of the different processes. One process step monitored and/or controlled may be the feed of input materials or the discharge of chemical products. Another process step monitored and/or controlled may be the allocation of environmental attributes to chemical products produced via the chemical production network. Yet another process step monitored and/or controlled may be the registration of environmental attributes associated with input material(s) entering the system boundary of the chemical production network. Yet another process step monitored and/or controlled may be the management of environmental attributes associated with input material(s) and chemical product(s) of the chemical production network.

The operating system may be configured to access data related the inputs material(s), the process(es) and/or the chemical product(s) produced by the chemical production network. The operating system may be configured to convert a recycled, renewable, or bio-based content of the one or more input material(s) used in the chemical production network to balancing units. The operating system may be configured to allocate the balancing units to at least one balancing account associated with the recycled or bio-based content of the input materials. The operating system may be configured to allocate at least a part of the balancing units from the at least one balancing account to the at least one chemical product.

The operating system may be configured to manage balancing units related to the input and chemical products produced by the chemical production network. In particular, the operating system may be configured to determine balancing units associated with the use of input materials impacting the environmental property/attribute of the chemical products produced by the chemical production network. The operating system may be configured to determine balancing units associated with the chemical product(s) and the environmental property of the chemical product(s). This way the operating system may be configured to allocate balancing units to balancing accounts or to deallocate balancing units from the balancing accounts. The balancing units may be viewed as a credit that may be deposited in an account (e.g., a digital inventory) or deducted from an account related to the input and chemical products of the chemical production network.

The operating system may be configured to register inbound environmental attributes, to convert the inbound environmental attributes to balancing units (and back as needed), and/or to assign outbound environmental attributes and to manage inbound allocation as well as outbound assignment.

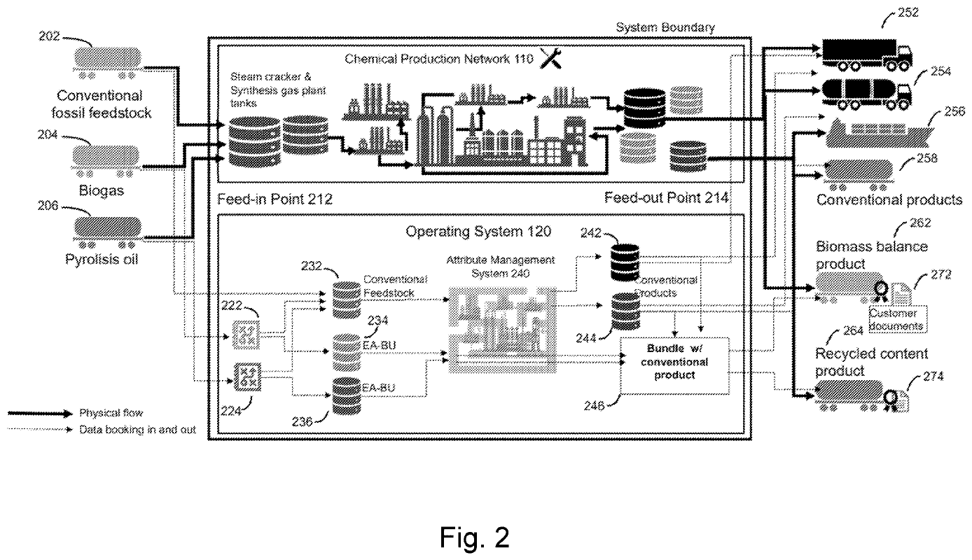

FIG. 2 illustrates an example of a chemical production network 110 producing one or more chemical product(s) from one or more input material(s) in connection with an operating system 120 including an attribute management system 240 to manage two or more environmental attributes. Chemical production network 110 is described above with reference to FIG. 1.

Operating system 120 is a digital operating system configured to collect, store, manage and interpret a wide range of production and/or business data for chemical production network 110. Operating system 120 may be part of an Enterprise Resource Planning (ERP) system. Alternatively, operating system 120 may be partly implemented in an ERP system and partly implemented in one or more additional systems coupled with an ERP system. Operating system 120 may also be implemented in one or more systems outside of an ERP system.

Input materials 202-206 are provided to chemical production network 110 at the feed-in point 212. The input materials may include conventional fossil feedstock 202 (e.g., naphtha) as well as sustainable input materials 204-206. The sustainable input materials 204-206 may include renewable input materials (such as biogas and/or bio-naphtha) and/or recycled input materials (e.g., pyrolysis oil). After they are delivered to chemical production network 110, the conventional input materials 202 and the sustainable input materials 204-206 may be combined (e.g., by being fed into the same tank) as they enter the chemical production process.

Input material data for sustainable input material 204 is provided to operating system 120 at 222. Similarly, input material data for sustainable input material 206 is provided to operating system 120 at 224. For example, the goods receipt (and/or a BOM and/or a chemical production recipe) including the input material data for each of the sustainable input materials may be electronically provided to operating system 120 when sustainable materials 204-206 are delivered to chemical production network 110. Operating system 120 may receive input material data 222-224 through an interface to a local or a remote database or an ERP system, in particular its supply chain module, or any computing system or apparatus, such as a centralized or decentralized computing system or apparatus including processing and storage. The input material data for each input material may hence be gathered from an ERP system or any computing system or apparatus, such as a centralized or decentralized computing system or apparatus including processing and storage. In some cases, the input material data of each input material is gathered through an interface to more than one database. It therefore may be necessary to convert the information retrieved from different databases into a single format to allow further processing. In particular, the input material data obtained from databases may be attributed to the input material via the identification of an input material in the database that has to be translated to the identification of the input material of the process data used in the process according to the present disclosure.

Operating system 120 may initiate a virtual production step after it receives the input material data for sustainable materials 204-206. Virtual production refers to receiving input material data for a sustainable input material and producing environmental attributes (based on the sustainable input material) and also “producing” conventional input material data (e.g., data describing the corresponding amount and/or value of the conventional input material).

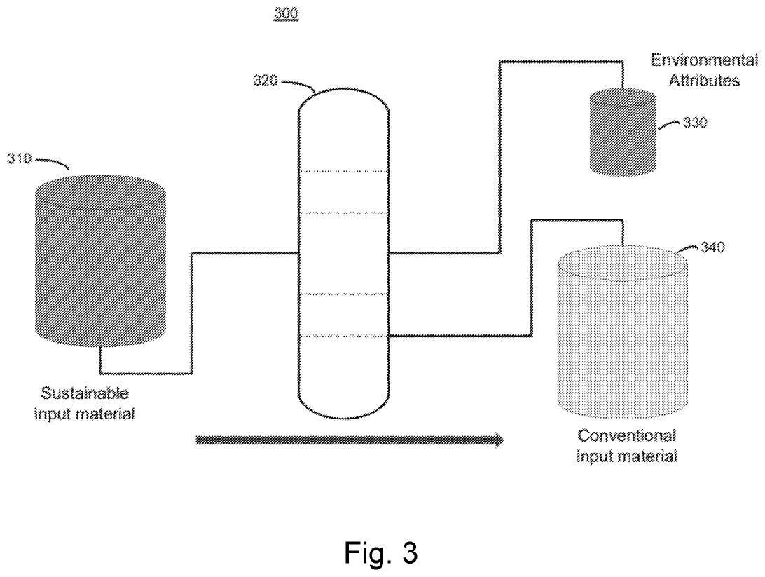

For example, with reference to FIGS. 2 and 3, operating system 120 initiates virtual production process 300 when it receives input material data for sustainable input material(s) (e.g., 222-224). Using input material data 222-224, virtual production process 300 may parse the input material data and apply a corresponding recipe. For example, virtual production process 300 may determine the volume (or mass) and type of sustainable input material that was received from the input material data. It may then apply virtual production step(s) 320 to the sustainable input material 310. Virtual production step(s) 320 may “produce” both environmental attributes 330 and conventional input material 340. The amount of conventional input material 340 (virtually) produced may be equal to the amount of sustainable input material 310.

Referring again to FIG. 2, after the virtual production process, operating system 120 may credit digital inventory (which may also be referred to as a virtual balancing account) 232 with the amount of conventional feedstock that was created by the virtual production process(es). Operating system 120 may also convert the environmental attributes to balancing units and allocate or credit those balancing units to digital BU inventories (or virtual balancing accounts) 234-236. The conversion may include a conversion factor that takes account of the chemical difference between fossil-based input materials, such as naphtha and methane, and non-fossil input materials, such as pyrolysis oil. The conversion factor may relate to the lower heating value of the pyrolysis oil in relation to the lower heating value of naphtha or methane. The conversion factor may include the ratio of the lower heating value of pyrolysis oil to naphtha or methane. This way the chemical difference between the fossil and the renewable input material can be taken into account.

Digital inventories 234-236 may determine and track both the amount (e.g., volume and/or mass) and the value of sustainable input material 204-206, respectively. For example, operating system 120 may parse input material data 222 to determine the amount of sustainable input materials 204 that was received. Similarly, operating system 120 may parse input material data 224 to determine the amount of sustainable input material 206 that was received. Operating system 120 may then credit digital inventories 234 and 236, respectively, with the amount of sustainable input materials that were received.

Operating system 120 may also determine a value associated with the balancing units it credits to digital inventories. For example, operating system 120 may compute the difference in cost between sustainable input materials 204-206 and corresponding equivalent fossil input materials to determine the value of the balancing units. Operating system 120 may use average price, actual price, market price or other suitable values to determine the cost of the equivalent amount of fossil input materials. Operating system 120 stores and tracks the amounts and values corresponding to sustainable input materials in digital inventories 234-236. For example, the balancing units stored in digital inventories 234-236 may include the amount and/or value information corresponding to sustainable inputs 204-206.

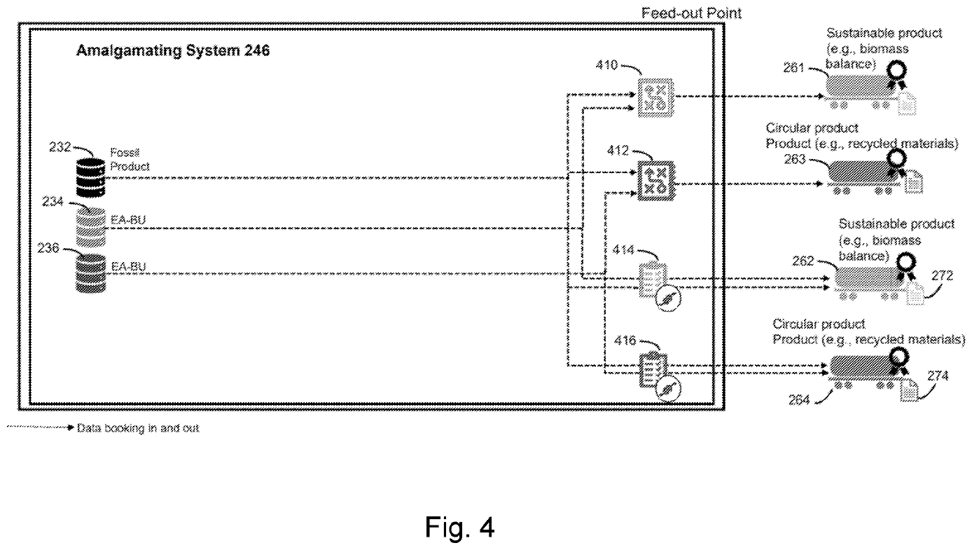

Operating system 120 includes amalgamating system 246 to create sustainable chemical products by combining balancing units with conventional products. With reference to FIGS. 2 and 4, operating system 210 processes an order for a product 252-264. If the customer purchased a conventional chemical product 252-258, operating system 120 may process the purchase using conventional product digital inventories 242-244.

If, however, the customer purchased a sustainable chemical product, operating system 120 may direct amalgamating system 246 to combine balancing units from digital inventories (or virtual balancing accounts) 234-236 with conventional products from digital inventories 242-246. Amalgamating system 246 may generate a digital asset (which may or may not be incorporated into another record such as a BOM and/or sales record) 272-274 that defines (or specifies) a sustainable product from the combination of balancing units and conventional products (e.g., using combining or bundling logic 410-416). For example, amalgamating system 246 may create a sustainable product by combining conventional products (from 242-244) with environmental attributes from digital inventory 234 as shown by 272. Similarly, amalgamating system 246 may create a circular product by combining conventional products (from 242-244) with environmental attributes from digital inventory 236 as shown by 274. Thus, operating system 120 enables chemical production network 110 to efficiently create multiple sustainable products from multiple input materials including sustainable input materials that are combined with fossil input materials in a large interconnected chemical production network.

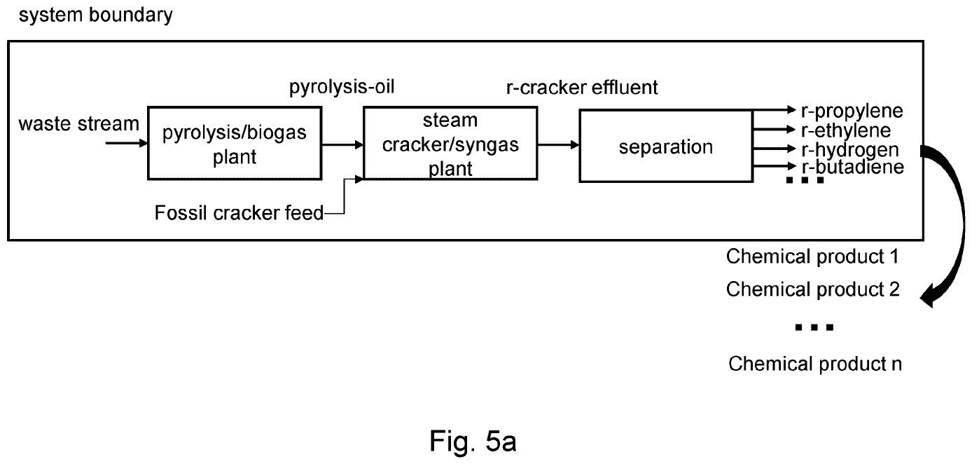

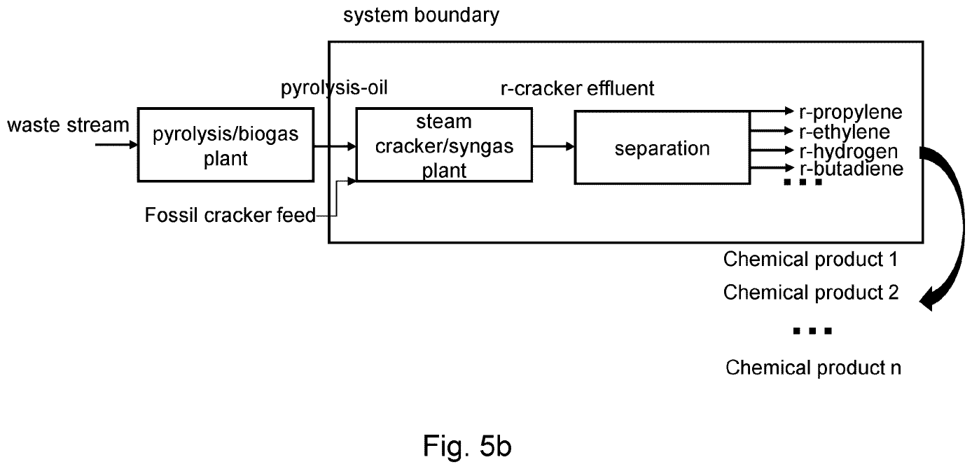

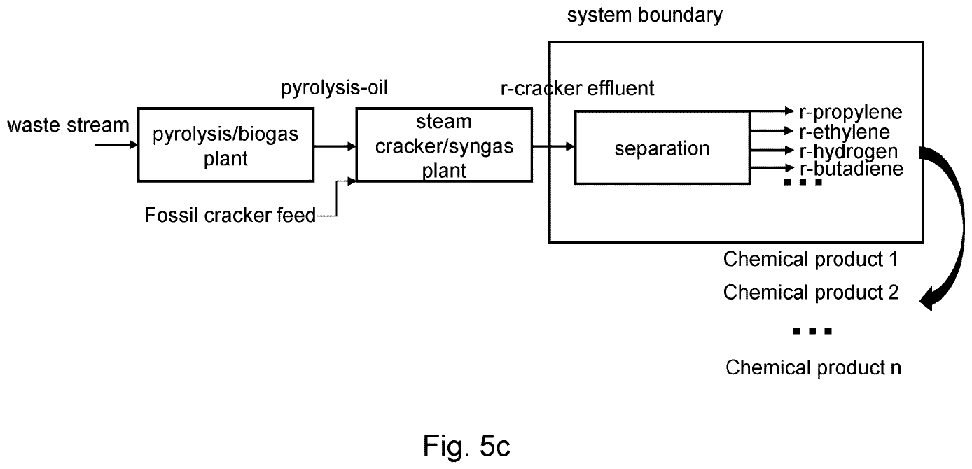

FIGS. 5a-c illustrate a part of a chemical production network producing multiple chemical product(s) from fossil and non-fossil input material(s).

The chemical production network may comprise a pyrolysis unit for pyrolysis of recycled waste. A waste stream may be fed to the pyrolysis unit. Waste stream may include plastics, rubber (including tires), textiles, wood, biowaste, modified celluloses, wet laid products, and any other material suitable for pyrolysis. The recycled waste stream may include a stream containing at least in part post-industrial, or post-consumer, or both post-industrial and post-consumer materials. A post-consumer material may be a material that has been used at least once for its intended application for any duration of time regardless of wear, or has been sold to an end use customer, or which is discarded by any person or entity other than a manufacturer or business engaged in the manufacture or sale of the material. A post-industrial material may be a material that has been created and has not been used for its intended application or has not been sold to the end use customer or discarded by a manufacturer or any other entity engaged in the sale of the material.

Examples of post-industrial materials include rework, regrind, scrap, trim, out of specification materials, and finished materials transferred from a manufacturer to any downstream customer (e.g., manufacturer to wholesaler to distributor) but not yet used or sold to the end use customer. The waste stream may be isolated as one type of waste stream with specific waste material, or it may be a stream of mixed wastes.

Examples of plastics as a waste stream include high density polyethylene and copolymers thereof, low density polyethylene and copolymers thereof, polypropylene and copolymers thereof, other polyolefins, polystyrene, polyvinyl chloride (PVC), polyvinylidene chloride (PVDC), polyesters including polyethylene terephthalate, co-polyesters and terephthalate co-polyesters (e.g. containing residues of 2,2,4,4-tetramethyl-1,3-cyclobutanediol, CHDM cyclohexanedimethanol, neopentyl glycol monomers, or propylene glycol), polyethylene terephthalate, polyamides, poly(methyl methacrylate), polytetrafluoroethylene, acrylonitrile butadiene styrene (ABS), polyurethanes, cellulosics and derivates thereof such as cellulose acetate, cellulose diacetate, cellulose triacetate, cellulose propionate, cellulose butyrate; regenerated cellulosics such as viscose and rayons, epoxy, polyamides, phenolic resins, polyacetal, polycarbonates, polyphenylene-based alloys, polypropylene and copolymers thereof, polystyrene, styrenic compounds, vinyl based compounds, styrene acrylonitrile, thermoplastic elastomers, urea based polymers and/or melamine containing polymers. The pyrolysis effluent exiting the pyrolysis unit may comprise pyrolysis oil. The pyrolysis oil may be used as feedstock for a cracker unit or a syngas plant. The pyrolysis oil may be fed to the cracker unit as recycled feedstock together with a non-recycle or conventional feedstock (e.g., propane, ethane, naphtha and/or natural gasoline). In addition, or alternatively to the recycled feedstock, bio-based feedstock may be fed to the cracker unit. For example, bio-naphtha may be produced through hydrogenation of bio-based material such palm oil and tallow and provided as feedstock to the cracker unit. In further embodiments pyrolysis wax, pyrolysis gas, pyrolysis char, or synthesis gas may be provided to the cracker unit as feedstock.

The cracker unit may include a steam cracker that breaks saturated hydrocarbons down into smaller, often unsaturated, hydrocarbons. Steam crackers are facilities in which fossil feedstock such as naphtha, liquefied petroleum gas (LPG), ethane, propane, or butane and/or non-fossil feedstock is thermally cracked through the use of steam in steam cracking furnaces or electric furnaces to produce lighter hydrocarbons.

The output effluent of the cracker unit may be a recycled and/or bio-based content effluent containing light olefins, C4 products and heavy cracker products such C5, C6, C7, C8, C9, C10 products or mixtures. The recycled and/or bio-based content cracked effluent may be subjected to separation in a separation train. The recycled or bio-based content effluent can be separated in different fractions containing recycled or bio-based content from the recycled or bio-based content of the cracked effluent.

The light olefins fraction may include ethylene and propylene. Ethylene may be used to produce polyethylene, ethylene chloride and ethylene oxide. Polyethylene, ethylene chloride or ethylene oxide may be chemical intermediates used to produce chemical product(s) that exit the chemical production network. Polyethylene, ethylene chloride or ethylene oxide may be chemical product(s) that exit the chemical production network. Polyethylene, ethylene chloride or ethylene oxide may be used for producing packaging as end product, for plastic processing or for producing end products in construction and textile production. Propylene may be used to produce polypropylene, propylene oxide, acrylic acid, or other chemical derivatives. Propylene and its derivatives may be chemical intermediates used to produce chemical product(s) that exit the chemical production network. Propylene and its derivatives may be chemical product(s) that exit the chemical production network. Propylene and its derivatives may be used for producing packaging as end product, for producing furniture as end products or for producing end products in automotive production.

The C4 fraction may contain a gas mixture comprising C4 olefins from which butadiene and isobutene may be extracted. The residue, a mixture of butene and butanes, may be used as chemical intermediate for further production processes of the chemical production network. Butadiene and its derivatives may be chemical intermediates used to produce chemical product(s) that exit the chemical production network. Butadiene and its derivatives may be chemical product(s) that exit the chemical production network. Butadiene and its derivatives may be used for producing end products such as tires, papers, plastics, rubber, petroleum, lube, or perfumes. Isobutene (Isobutylene) and its derivatives may be chemical intermediates used to produce chemical product(s) that exit the chemical production network. Isobutene (Isobutylene) and its derivatives may be chemical product(s) that exit the chemical production network. Isobutene (Isobutylene) and its derivatives may be used for producing butyl rubber and for polyisobutylene for end products such as tires, papers, plastics, rubber, petroleum, lube, or perfumes.

The heavy cracker fraction may contain 5-12 hydrocarbon atoms (e.g., C5 non-aromatics, C7/C8 mixtures, C9). C5 non aromatics may be chemical intermediates used to produce further chemical intermediate(s) such as cyclopentane-and n/i pentane mixtures or chemical product(s) that exit the chemical production network. C9 fraction, not hydrogenated or hydrogenated, C7/8 mixtures or xylol-mixtures may be chemical intermediates used to produce further chemical intermediate(s) such as hydrocarbon resins, used as a blending component for premium gasoline or used for the production of benzene.

Residues like pyrolysis oil from ethylene production may be used as chemical intermediate for the production of carbon black, as an auxiliary material in the chemical industry, as a raw material for distillation of naphthalene and indene.

Based on the recycled and/or bio-based content of the cracked effluent, the fractions from the cracked effluent and any chemical products produced from any component of such effluent may contain recycled and/or bio-based content. For example, the light olefin fraction may contain recycled and/or bio-based content. Further for example, the ethylene or propylene fraction may contain recycled and/or bio-based content. Further for example, chemical products produced from the ethylene or propylene fraction may contain recycled and/or bio-based content. This way the recycled and/or bio-based content may be contained in the produced chemical intermediates, chemical product(s), or end products. As shown in FIG. 5 “r-ethylene” (recycle content ethylene) may be a composition comprising: (a) ethylene obtained from cracking of a cracker feed containing pyrolysis oil, or (b) ethylene having a recycled content value attributed to at least a portion of the ethylene. Similarly, the “r-propylene” (recycle content propylene) can be a composition comprising (a) propylene obtained from cracking of a cracker feed containing pyrolysis oil, or (b) propylene having a recycled content value attributed to at least a portion of the propylene. Both, r-ethylene and r-propylene can be summarized under the term r-olefin (recycle content olefin).

FIG. 5a illustrates an embodiment for a system boundary of the chemical production network, which includes the pyrolysis step. The waste stream forms the entry point into the chemical production network. The chemical end products form the exit point out of the chemical production network. FIG. 5b illustrates another embodiment for the system boundary of the chemical production network, which excludes the pyrolysis step. The pyrolysis oil and the fossil feed form the entry point into the chemical production network. The chemical end products form the exit point out of the chemical production network. FIG. 5c illustrates another embodiment for the system boundary of the chemical production network, which excludes the pyrolysis step and the cracking step. The cracker effluent produced at least in part from pyrolysis oil forms the entry point into the chemical production network. The chemical end products form the exit point out of the chemical production network. The chemical production networks and the system boundaries illustrated in FIGS. 5a to 5c are examples and should not be considered limiting.

In the examples shown in FIGS. 5a to 5c, the recycled and/or bio-based content may be the environmental attribute of the respective input material. In FIG. 5a, the waste stream is the input material to the chemical production network including the pyrolysis unit. The environmental attribute associated with such waste stream may include the environmental attribute type such as recycled and the waste type such as mixed plastics waste. The environmental attribute may be separated from the material flow through the chemical production network on entry of the waste stream. The environmental attribute may be allocated to the virtual balancing account associated with the respective environmental attribute. In FIG. 5b, the pyrolysis oil is the input material to the chemical production network excluding the pyrolysis unit. The environmental attribute associated with such pyrolysis oil may include the environmental attribute type such as recycled, the material type such as pyrolysis oil and the waste type such as mixed plastics waste. The environmental attribute may be separated from the material flow through the chemical production network on entry of the pyrolysis oil to the chemical production network. The environmental attribute may be allocated to the virtual balancing account associated with the respective environmental attribute. In FIG. 5b, the cracked effluent produced from pyrolysis oil is the input material to the chemical production network excluding the pyrolysis unit and the cracker unit. The environmental attribute associated with such effluent may include the environmental attribute type such as recycled, the material type such as effluent produced from pyrolysis oil and the waste type such as mixed plastics waste. The environmental attribute may be separated from the material flow through the chemical production network on entry of the cracked effluent to the chemical production network. The environmental attribute may be allocated to the virtual balancing account associated with the respective environmental attribute. By separating the environmental attribute from the material flow of the chemical production network, a reliable and simple assignment of environmental attributes to the chemical products produced by the chemical production network can be realized. By way of virtual balancing accounts, the environmental attributes can be collected on entry to the chemical production network. On exit of chemical products, the environmental attributes collected on entry can be assigned to such products and de-allocated from the virtual balancing account. Thus, it can be ensured 1) that only environmental attributes are assigned to chemical products that entered the chemical production network and 2) that are not already assigned to another chemical product. As a result, the positive environmental impact is only counted once and can be tracked from entry to exit of the chemical production network.

In the context of this example a pyrolysis unit followed by a steam cracker unit is shown. This is not to be considered limiting. The concepts disclosed herein equally apply to equivalent chemical value/production chains such syngas plant-based value/production chains.

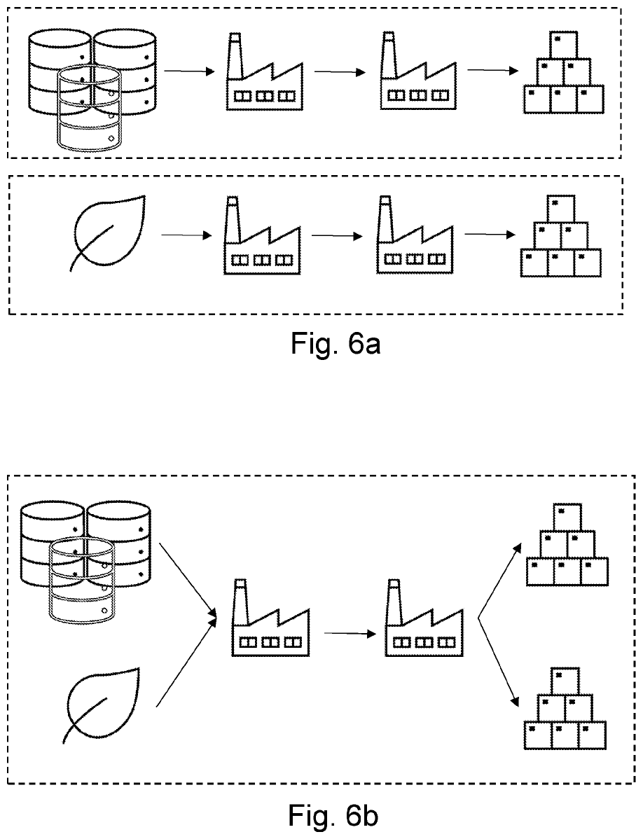

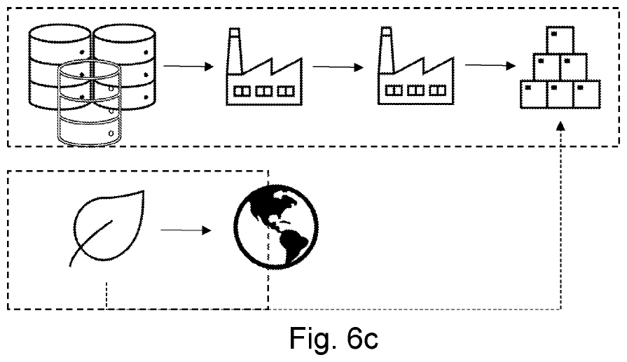

FIG. 6a to c illustrate examples of allocation schemes allocating the use of renewable or bio-based input materials to chemical products of the chemical production network.

As illustrated in FIGS. 1-5 chemical production networks can comprise complex interconnected production sites that chemically convert one or more input materials via chemical processing to one or more chemical products. To account for the use of recycled or renewable content in chemical production, allocation rules may be used. This way recycled, renewable or bio-based content of input materials may be allocated to chemical products. The renewable content may be based on input material from renewable sources. The renewable content may comprise bio-based input materials produced from living organisms such as different types of crops, wood, or algae. The recycled content may comprise any recycled material used in production of new materials. This may include any recycled bio-based or bio-based materials e.g., as produced from chemical or mechanical recycling.

Accounting principles for allocating the use of recycled or renewable content are for example defined in ISO 22095. Four different chain of custody models may be used: Identity preservation models, segregated models, mass balance models, or book and claim models.

FIG. 6a illustrates an example of a dedicated or segregated production network. The production network comprises a first production chain for producing the chemical product(s) from fossil material(s) and a second production chain for producing the chemical product from bio-based input material(s). The first and the second production chain are not interconnected. The first and the second production chains produce fossil-based and bio-based or recycled chemical product(s), respectively. Example for such dedicated production environments include fermentation or chemical transformation, such as polyethylene production from sugar cane, bio-poly lactic acid (PLA) production from corn, bio-succinic acid, or bio-butanediol (BDO).

FIG. 6b illustrates an example of a complex production network. In contrast to the production network of FIG. 6a the fossil-based input material(s) are co-fed and mixed with bio-based or recycled input materials. The production network produces via one or more chemical process chain(s) with intermediates one or more material outputs or products. For the sake of simplicity FIG. 6b illustrates the mass balancing approach for one chemical process chain producing one chemical product or product. In the mass balancing model, the physical mixing or co-feeding of bio-based or recycled input material with conventional fossil input materials is accounted for. Here the feed into the production network and the feed of output products form a system boundary. The mass balance of input and chemical products connects the used bio-based or recycled input material to the produced output product. Mass balance allows to keep track of the total amount of input material (e. g. recycled or bio-based or bio-based materials) throughout the production network and allows for allocation to chemical products. Materials with different sets of specified characteristics may be mixed. E.g. recycled or bio-based feedstock replaces an equivalent amount of fossil feedstock at the beginning of the value chain (input material) and is allocated to a product (chemical product) in such a manner that the input and output match. For this model, the proportion of the input with specified characteristics might only match the initial proportions on average and will typically vary across different outputs. This means that e.g., recycled and fossil input materials are mixed and that the chemical or technical proportions in each chemical product are not tracked.

Mass balance may include conversion factors to ensure the amount of input material is correlated with the amount of chemical product. The calculation may be made over a pre-defined or specified time period. Mass balance may be based on a balancing unit such as mass, energy, or carbon.

FIG. 6c illustrates a complex production network associated with a book and claim scheme. In a book and claim scheme, the characteristic renewable or recycled input material is not linked to the actual material flows. Book & Claim allows to de-couple a specific characteristic, such as renewable, from the physical product and to transfer the characteristic separately via a dedicated registry in the form of a digital asset. This approach may be used for renewable energy. Book and claim may be based on a book and claim accounting unit such as kilowatthours for electricity.

In identity preservation models or segregated approaches as illustrated in FIG. 6a renewable, recycled or bio-based input materials may not be mixed with fossil input material. In mass balance or book and claim approaches as illustrated in FIGS. 6b and 6c renewable or recycled or bio-based materials with fossil input material may be mixed. In view of the increasing number of different sources for more sustainable chemical production and the number of allocation schemes, an efficient and robust operation system for operating complex production networks like chemical production networks is required.

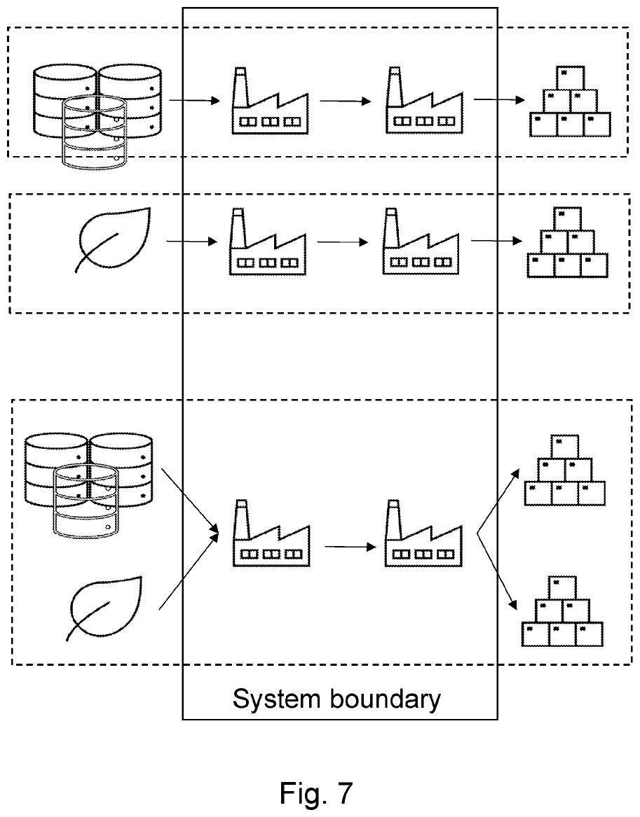

FIG. 7 illustrates an example of a chemical production network with different allocation schemes.

The chemical production network may include multiple production chains with different allocation schemes. The system boundary of the chemical production network may be defined by the entry points to the chemical production network and the exit points from the chemical production network. The production chains may be defined by the chemical product(s) produced via such production chains. The production chain logic may be based on process data associated with process steps from input material(s) to chemical product(s). For each production chain an allocation scheme may be applicable (and the application allocation scheme may be assigned to the production chain). In addition for each production chain a balancing system (e.g., a virtual balancing account) may be applicable or the production chain logic may be embedded in the attribution rules.

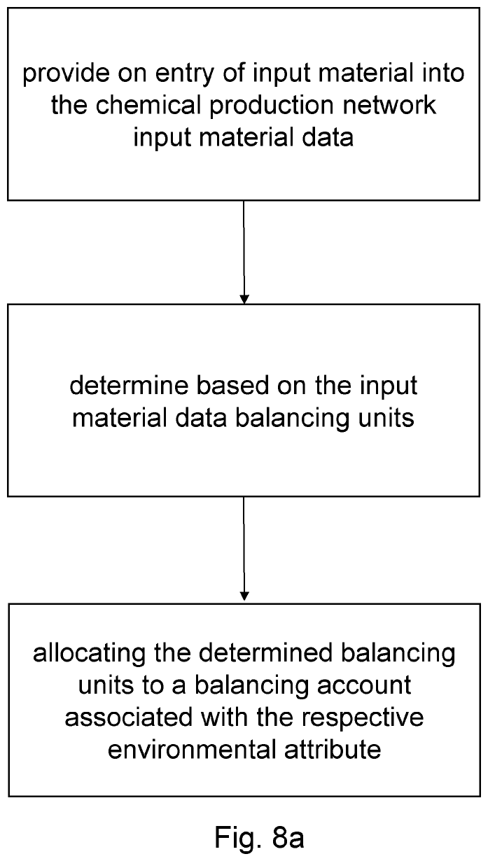

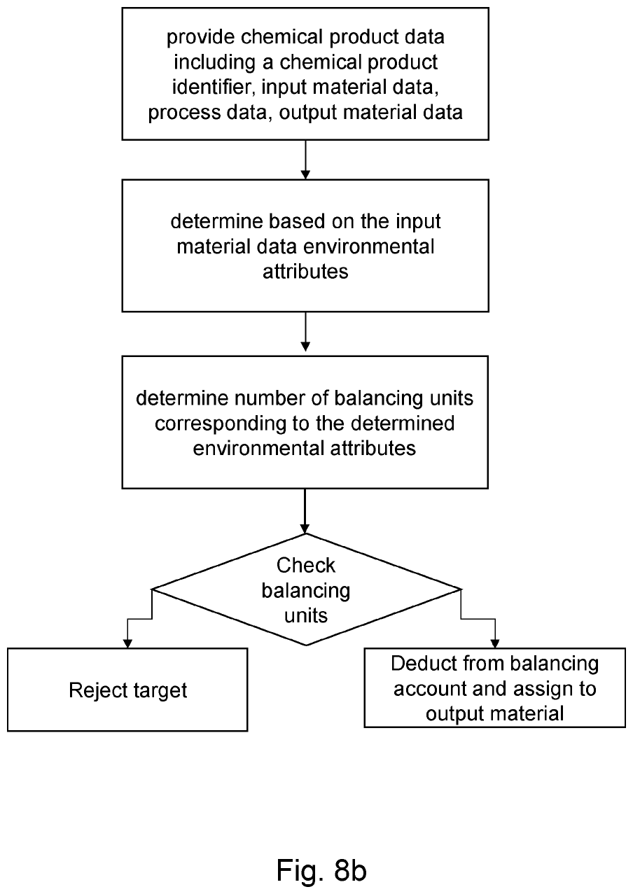

FIG. 8a, b illustrate inbound and outbound allocation of environmental attributes for a chemical production network.

FIG. 8a illustrates an example of an inbound allocation process for the chemical production network.

As for example illustrated in FIG. 1, the chemical production network may be operated by the operating system configured to register inbound environmental attributes, to assign outbound environmental attributes and/or to manage inbound registration as well as outbound assignment via allocation rules. The allocation rules may include allocation schemes as illustrated in FIGS. 6a-c. The chemical production network may include the network as for example described in the context of FIG. 5 or 7.

On input material(s) entering the chemical production network or the physical system boundary of the chemical production network the inbound registration or allocation takes place. In this context input materials signify any materials that enter the system boundary of the chemical production network and are used to produce chemical product(s) leaving the chemical production network. Such input material(s) may be associated with an input material identifier. Possible identifiers include decentral identifiers, input material specification(s), input material order number(s), input material LOT number(s), input material batch number(s), input material supplier specification(s) or combination thereof.

On entry of the input material(s) to the chemical production network, input material data may be provided. The input material data may include input material identifier(s), input material amount, input material value, and/or one or more environmental attribute(s) associated with the input material(s).

Based on the input material data, in particular the input material amount, input material value, and/or one or more environmental attribute(s) associated with the input material, balancing units may be determined by converting the input material amount (and/or input material value) to balancing units for the respective environmental attribute. The balancing units may relate to mass, weight, carbon atoms, hydrogen atoms, methane equivalents, energy property or any other suitable measure to quantify the environmental impact of the environmental attribute.

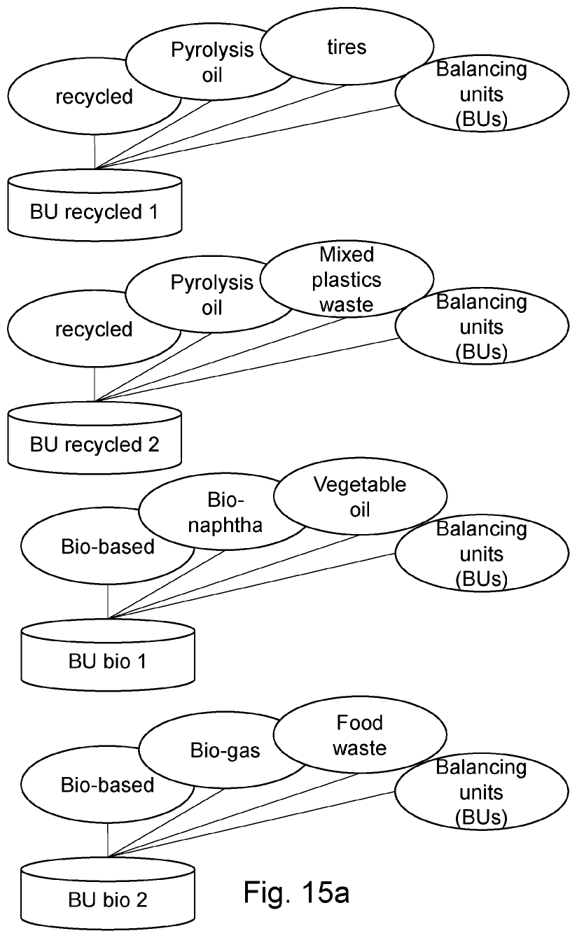

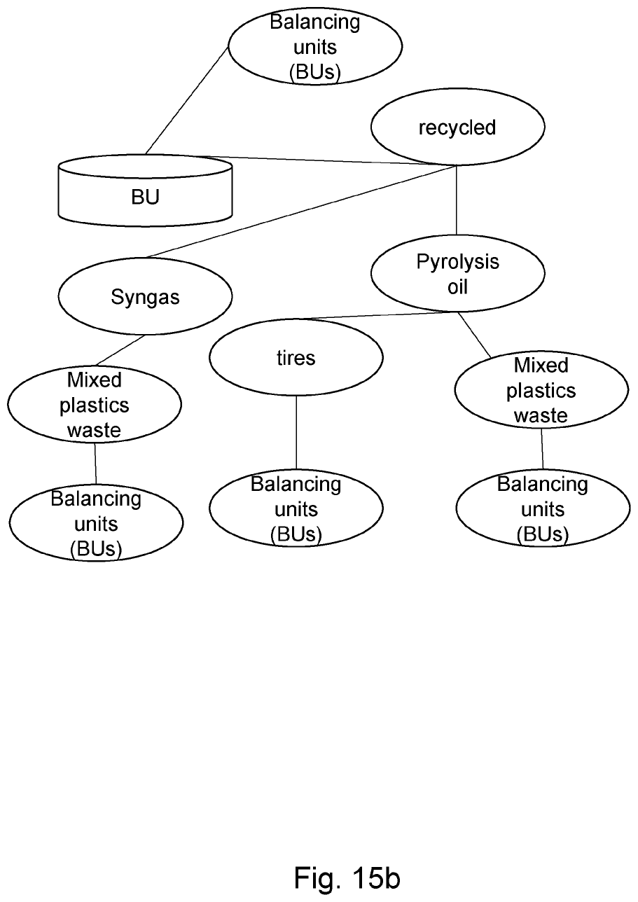

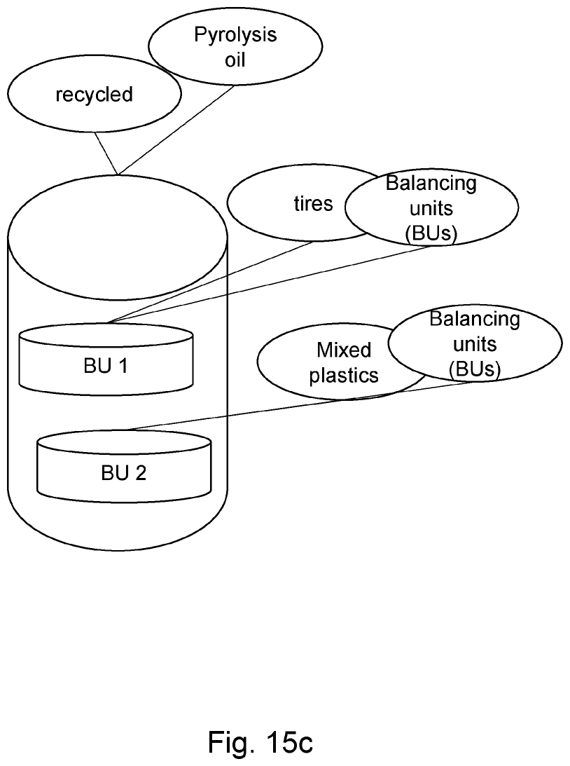

The balancing units may be allocated to a balancing account associated with the respective environmental attribute. This way the environmental attributes associated with input material(s) entering the chemical production network can be tracked or stored in an accounting system. An example of a virtual accounting system is shown in FIGS. 15a-c, where each balancing account is associated with metadata signifying the input material such as pyrolysis oil, environmental attribute type such as recycled and waste type such as tires.

For allocation the input material data may include respective metadata for the input material. Allocation rules may include instructions to allocate the environmental attribute(s) included in the input material data to the respective balancing account. In the inbound process such allocation rules may be used to allocate environmental attributes from the input material data to the virtual accounting system.

For example, the method for allocating balancing units may be associated with pyrolysis oil as input material to a complex chemical production network.

First the amount and type of waste stream may be provided to a computing interface configured to allocate the environmental attribute. The quantity and type of waste stream may be stored in connection with the pyrolysis plant(s) and pyrolysis oil produced by the plant. For instance, a pyrolysis oil producer may operate multiple plants and may track the amounts and type of waste streams fed to the plants. The waste may be provided to the pyrolysis plant separately according to type of waste or the waste may be mixed. Based on such tracking the quantity of pyrolysis oil and type of waste stream may be allocated to the produced pyrolysis oil.

The amount of pyrolysis oil and the type of waste stream may be provided to the operating system of a complex chemical production network. Based on the amount of pyrolysis oil balancing units may be generated. Such generation may include a conversion factor that takes account of the chemical difference between fossil-based input materials, such as naphtha and methane, and non-fossil input materials, such as pyrolysis oil. The conversion factor may relate to the lower heating value of the pyrolysis oil in relation the lower heating value of naphtha or methane. This way the chemical difference between the fossil and the recycled input material can be taken into account. Pyrolysis oil may be mixed with fossil input material and the fraction of pyrolysis oil may be registered in the account for balancing environmental units.

The balancing units may be allocated to the respective balancing account(s). For example, the allocation rule may be associated with balancing account 1 of FIG. 15a. In such a case the balancing units for the pyrolysis oil produced from waste of the waste stream type tires and used in a specified amount in the production network will be allocated to balancing account 1. On production of the chemical products such balancing units may be allocated to the chemical product according to the allocation rule. The allocation rule that specifies free attribution between production processes of the chemical production network may provide for reliable allocation in complex production networks. In such case the allocation of balancing units may be decoupled from the material flows of the chemical production network.

FIG. 8b illustrates an example of an outbound allocation process for the chemical production network.

As for example illustrated in FIG. 1, the chemical production network may be operated by the operating system configured to register inbound environmental attributes, to assign outbound environmental attributes and/or to manage inbound registration as well as outbound assignment via allocation rules. The allocation rules may include allocation schemes as illustrated in FIGS. 6a-c. The chemical production network may include the network as described in the context of FIG. 5 or 7.

The chemical product may be produced from input material that entered the chemical production network. Chemical product data including the chemical product identifier may be provided. The chemical product data may further include input material data associated with the input materials used to produce the chemical product(s), process data associated with the production chain for producing the chemical products and/or data related to the chemical product data, such as a chemical product specification or a chemical product amount.

Based on the input material data environmental attributes associated with the input material(s) used to produce the chemical product(s) may be determined. The input material data may specify the total amount of input material(s) that entered the production chain for producing the amount of chemical product. The input material data may further specify the environmental attributes available for the respective input material(s). From the total amount of input material(s), the number of balancing units for respective environmental attributes available for such input material(s) may be determined. This way the maximum number of balancing units for the respective environmental attribute attributable to the chemical product may be determined.

Based on the environmental attribute(s) attributable to the chemical product, the number of balancing units corresponding to the determined environmental attributes may be determined. The determined balancing units may be compared to balancing units stored in balancing account(s) for the respective environmental attribute(s).

If the balance of the respective balancing account(s) for the respective environmental attribute(s) is not sufficient, the environmental attribute(s) is rejected. If the balancing units are available, the balancing units are deducted from the respective balancing account(s) and the environmental attribute(s) are assigned to the chemical product.

An example of an accounting system is shown in FIGS. 15a-c, where each balancing account is associated with metadata signifying the input material such as pyrolysis oil, environmental attribute type such as recycled and waste stream type such as tires. For assignment of the environmental attribute(s) to the chemical product, allocation rules may be defined. Allocation rules may include instructions to assign the environmental attribute(s) included in the input material data from the respective balancing account to the chemical product(s). For the outbound process such allocation rules may be used to assign environmental attributes from the virtual balancing system to the chemical product(s).

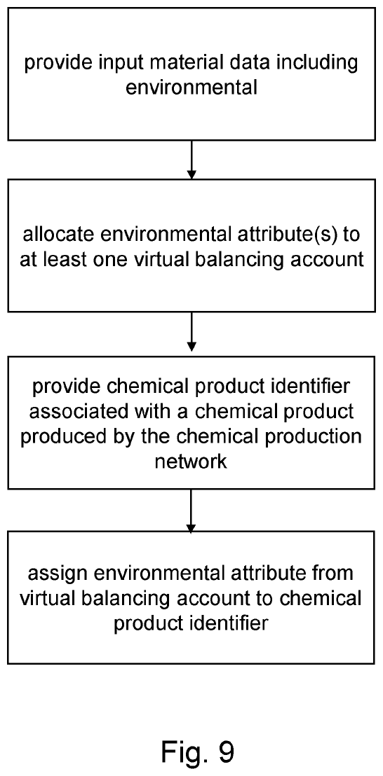

FIG. 9 illustrates an example of a method for assigning at least one environmental attribute to the least one chemical product produced by a chemical production network.

The one or more input material(s) may be provided at entry point(s) of the chemical production network. Material data related to the one or more input material(s) and respective environmental attributes may be provided to a computing interface configured for allocation and/or assignment of environmental attributes. The material data may be provided on, prior or after entry of the one or more input material(s) to the chemical production network. The material data may include the input material identifier associated with the respective input material(s) provided to the chemical production network. The input material identifier may be associated with the physical entity of the input material(s). This way the virtual identifier of the input material may be uniquely linked to the physical input material(s). Such linking may include a physical or virtual link of identifiers uniquely associated with the physical input material(s). For physical linking a tag or code may be physically connected to the material, e.g., by printing a QR code on the packaging. For virtual linking different identifiers associated with the physical material may be linked. For example, an order number, a batch number, LOT number or a combination thereof may be linked. The material data may include at least one environmental attribute associated with the respective input material and an amount of input material provided to the entry point of the chemical production network.

The one or more environmental attribute(s) may be allocated to at least one balancing account associated with the respective environmental attribute. The balancing account may be associated with a digital storage structure that stores data related to environmental attributes. The account may be associated with metadata identifying the account. The account may be associated with metadata identifying the environmental attributes and the environmental or balancing units allocated to the account. The account may be associated with metadata identifying the production chain the account is associated with. The account may be associated with metadata identifying the input or chemical product the account is associated with. The account may be part of a virtual accounting system including multiple accounts. The account may hold environmental attributes for transaction.

Environmental attributes may be allocated, added, deleted, withdrawn, deallocated, or deducted from the account. The virtual balancing account as for instance illustrated in FIG. 15c may by associated with environmental attribute types such as recycled or renewable. The virtual balancing account may by associated with input material types such as pyrolysis oil, bio-naphtha, bio-methane, or the like. The virtual balancing account associated with the environmental attribute type recycled may be further associated with the waste stream type such as mixed plastics waste, specific end product waste, e.g., tires waste or foam waste, post-consumer waste, pre-consumer waste or the like. The virtual balancing account may by associated with an allocation scheme such as segregated allocation, non-segregated allocation e.g., book and claim, mass balance with free attribution, mass balance without free attribution or combinations thereof.

The chemical product identifier associated with a chemical product produced by the chemical production network and provided at the exit point from the chemical production network may be provided. The chemical product identifier may be associated with the physical entity of the chemical product. This way the virtual identifier of the material may be uniquely linked to the physical material. Such linking may include a physical or virtual link of identifiers uniquely associated with the physical material. For physical linking a tag or code may be physically connected to the material, e.g., by printing a QR code on the packaging. For virtual linking different identifiers associated with the physical material may be linked. For example, an order number, a batch number, LOT number or a combination thereof may be linked.

At least one environmental attribute from the at least one balancing account associated with the respective environmental attribute may be assigned to the at least one chemical product identifier.

This way the chemical product may be provided together with the digital asset of the environmental attribute and the positive environmental impact associated with the chemical product can be tracked through the value chain using such chemical product. The tracking may stretch as far as the end product produced from the chemical product, re-use of the end product or recycling of the end product.

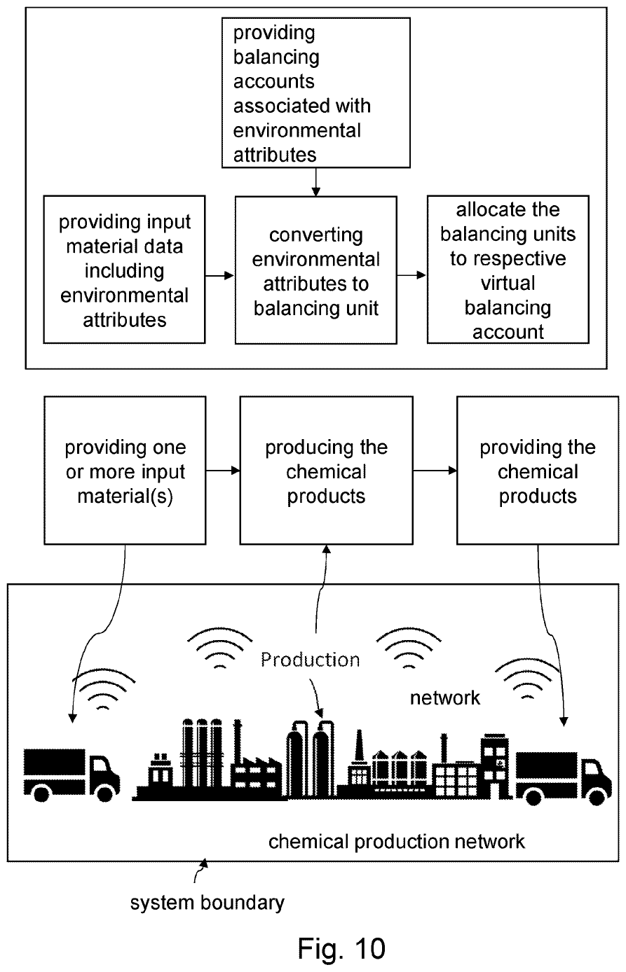

FIG. 10 illustrates an example of an apparatus for producing at least one chemical product associated with one or more environmental attribute(s) including an example method for converting and allocating to the virtual balancing account on inbound. The input materials such as pyrolysis oil, bio-naphtha or bio-gas may be provided to the chemical production network. The input materials may enter the system boundary of the chemical production network at the entry point, such as a such as a steam cracker or a syngas plant. The input materials may be used in the chemical production network to produce one or more chemical product(s) from the input materials. Chemical product(s) may be provided on exit points of the chemical production network. Chemical products may be MDI, TDI, PA6, EPS, PC, Polyols, Caprolactam, adipic acid, HMD, Polyamides.

On entry of the input material, the corresponding input material data may be provided via a network to the interface of the operating system. A data provider may be configured to provide material data related to the one or more input material(s) and respective environmental attributes to a computing interface configured to allocate the environmental attributes. The material data may be provided on, prior to or after providing the one or more input material(s) at entry points to the chemical production network. The input material identifier may be associated with the physical entity of the input material entering the chemical production network.

The data related to the input material identifier may include the environmental attribute associated with the respective input material, the amount of input material and the certificate certifying the environmental attribute. The amount of input material may be a measured amount of input material fed to a plant or storage of the chemical production network for producing one or more chemical product(s) from the input material(s). The input material identifier associated with the respective input material, the environmental attribute(s) associated with the respective input material(s) and the amount of input material(s) provided to the chemical production network may be provided. Such data may be provided via a communication network on entry to the system boundary, or the data may be transferred from a computing system to the operating system.

An inbound allocator may be configured to convert and allocate the one or more environmental attribute(s) to at least one balancing account associated with the respective environmental attribute. For allocation the one or more environmental attribute(s) may be converted to balancing units and the balancing units may be allocated to the balancing account. The one or more balancing unit(s) may be allocated to the at least one balancing account associated with the respective environmental attribute. The conversion may be based on a conversion factor such as mass, weight, carbon atoms, hydrogen atoms, methane equivalents or any other suitable measure for quantifying the environmental impact of the environmental attribute. By using balancing unit(s) and conversion it can be ensured that environmental attributes of input materials are only used once for assignment to chemical products. This way double counting on input or output is avoided and the positive environmental impact can be reliable assigned to chemical products.

Recycled, renewable or bio-based content used in the chemical production of one or more chemical product(s) may be converted to balancing units and allocated to balancing accounts to track such content. For the conversion of recycled, renewable, or bio-based content, input material data associated with input material(s) and including environmental attribute(s) may be provided. The input material data may relate to input material impacting the environmental property of the chemical product(s) produced by the chemical production network such as recycled or bio-based input material(s). The input material data may relate to the amount of input material impacting the environmental property of the chemical product(s) produced by the chemical production network. The amount may be provided as absolute value e.g., in kt per specified time frame such as kt/a. The quantity may be provided as relative value e.g., in % of the input material(s).

The input material data may relate to an energy value of the input material impacting at least one environmental property of the chemical production network or the energy value may be provided via a data base. The input material data may indicate an availability of input material impacting at least one environmental property of the chemical products produced by the chemical production. As an example, the input material data may include:
Input material 1 cracker: bio-naphtha
Input material quantity: x %.
Input material stock: x kt
Input material LHV: x MJ/kg
Input material 2 cracker: pyrolysis oil
Input material quantity: x %
Input material value: x €
Input material stock: x kt
Input material LHV: x MJ/kg

The one or more environmental attribute(s) may be converted to one or more balancing unit(s). In the example above, the environmental attribute associated with the input material 1 may be bio-based and the environmental attribute associated with input material 2 may be recycled. The conversion of the environmental attribute to balancing units may be based on the conversion factor relating conventional input material(s) to input material(s) associated with one or more environmental attribute(s). In the example above, the conversion factor may relate conventional naphtha based to bio naphtha or pyrolysis oil. The conversion factor may relate to the use of conventional input material(s) to the use of input material(s) associated with one or more environmental attribute(s) e.g., via mass or energy property. The conversion factor may hence take into account the difference between producing chemical products from conventional input material(s) and producing chemical products from non-conventional input material(s) or producing chemical products from a mix of conventional and non-conventional input materials. The conversion factor may relate to differences in chemical and/or physical properties of conventional and non-conventional input material(s).

In chemical production networks multiple value chains may be linked. Additionally different input materials impacting the environmental property of chemical products produced by the chemical production network may be used. Owing to the processing of chemicals in continuous or semi-continuous production and the complexity of chemical production networks, traceability of the input materials through the network may be hampered. In such scenarios an equivalent amount of input materials impacting the environmental property of chemical products produced by the chemical production network may be allocated to the virtual balancing account and assigned to one or more chemical product(s) of the chemical production network. This way certain input materials associated with at least one environmental attribute may replace an equivalent amount of fossil input materials and may be allocated to the virtual account or assigned to one or more chemical product(s). The environmental attributes may hence be decoupled from the physical material flow inside the chemical production network. Decoupling may be based on the mass balance model in that the equivalent amount assigned to the one or more chemical product(s) may not exceed the equivalent amount provided by input materials. If an equivalent amount has been allocated to the virtual account of one environmental attribute type, it may not be allocated a second time to another virtual account of the one environmental attribute type. Environmental attribute types may be recycled, bio-based, renewable or the like.

The most common examples of input materials impacting at least one environmental property of chemical products produced from such input materials are recycled, renewable or bio-based input materials. These may be used as feedstock for a cracker such as a steam cracker that breaks saturated hydrocarbons down into smaller, often unsaturated, hydrocarbons for producing the lighter alkenes such as olefins, including ethene (or ethylene) and propene (or propylene).

Recycled, renewable or bio-based input materials may have the same chemical properties as their fossil counterparts. They may be totally interchangeable and the input material quantities of e.g., naphtha or natural gas, may be substituted with the quantities of input materials associated with environmental attributes. For instance, Naphtha is commonly cited with a lower heating value (LHV) of about 44.3 MJ/kg and bio-naphtha is commonly cited with a similar LHV. In such scenario the energy based balancing unit may be about 1, meaning one unit naphtha corresponds to one unit bio-naphtha. The equivalent amount of input materials associated with the environmental attribute may be allocated in one-to-one correspondence.

However, some recycled, renewable, or bio-based input materials may have different carbon content, energetic value, or other chemical properties different to the fossil feedstocks to be replaced. This may lead to higher or lower requirements for recycled, renewable or bio-based input materials compared to the quantities of fossil feedstocks. In such situations, equivalent quantities of recycled, renewable, or bio-based input materials may not adhere to a one-to-one correspondence. For instance, a conversion factor based on the lower heating value (LHV) of fossil and bio-feedstocks as an approximation of the chemical properties may be used. Bio-gas is commonly cited with an LHV of 50 MJ/kg. In such scenario the balancing unit may be about 0.88, meaning one unit naphtha corresponds to 0.88 unit bio-naphtha. Hence the equivalent amount of input material(s) associated with at least one environmental property may be allocated according to the conversion factor of 0.88 in this scenario.

Different approaches exist to determine the conversion factor from conventional input material unit to balancing unit or equivalent assignable to one or more chemical products from chemical production. Conversion may depend on
mass such as mass of input material, mass equivalent of a molecular unit such as methane or a molecular weight,
an element, such as a number or a mole of one or more element(s) or molecular unit(s) or
an energy property such as heating value like lower or higher heating value.

By way of balancing units, the environmental attribute associated with the input material may be allocated to the virtual balancing account associated with the respective environmental attribute. For allocation, allocation rules mapping the environmental attribute to the virtual balancing account may be provided. The allocation rules may depend on the input material type and the environmental attribute type. The allocation rule may be linked to metadata relating to input material type and environmental attribute type. Similarly, the virtual balancing account may be linked to metadata relating to input material type and environmental attribute type. Based on the input material type and the environmental attribute type, the virtual balancing account the respective metadata may be matched, and the input material account may be selected for allocation.

The balancing units may be allocated to the selected virtual balancing account. For example, the allocation rule may be associated with the account for pyrolysis oil. On production of the chemical products corresponding balancing units may be assigned to the chemical products. The allocation rule may specify the assignment of balancing units to the chemical product.

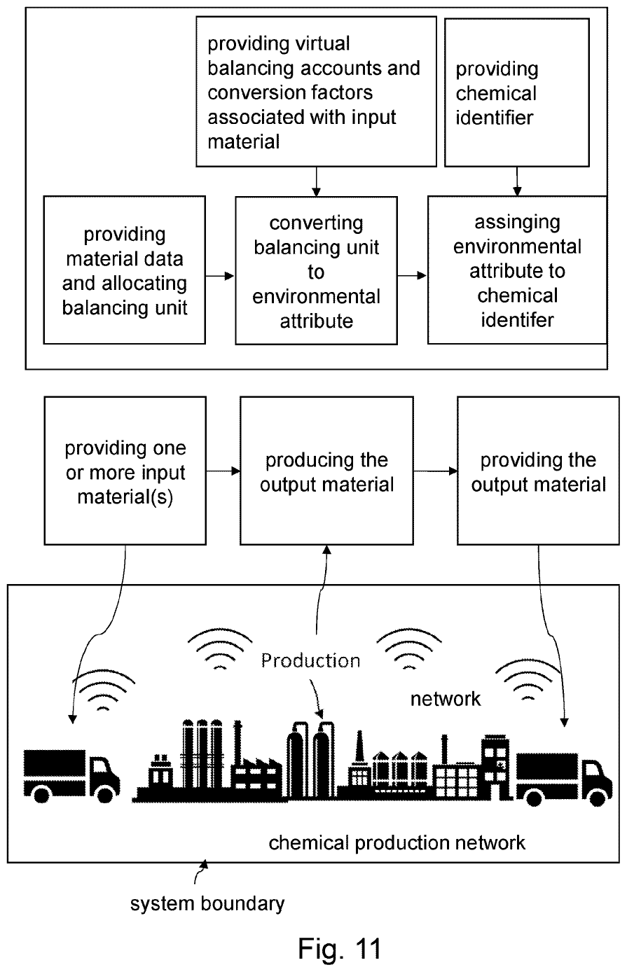

FIG. 11 illustrates an example of an apparatus for producing at least one chemical product associated with one or more environmental attribute(s) including an example method for assigning to the chemical product on outbound.

Similar to FIG. 10, the input materials such as pyrolysis oil, bio-naphtha or bio-gas may be provided to the chemical production network. The input materials may enter the system boundary of the chemical production network at the entry point, such as a such as a steam cracker or a syngas plant. The input materials may be used in the chemical production network to produce one or more chemical product(s) from the input materials. Chemical product(s) may be provided on exit points of the chemical production network. Chemical products may be MDI, TDI, PA6, EPS, PC, Polyols, Caprolactam, adipic acid, HMD, Polyamides.

Similar to FIG. 10, the one or more environmental attribute(s) may be converted and allocated to the virtual balancing account on inbound.

An identifier provider may be configured to provide the chemical product identifier associated with a chemical product produced by the chemical production network and provided at the exit point from the chemical production network. The chemical product(s) may be any chemical product produced by the chemical network.

An outbound assigner may be configured to assign at least one environmental attribute from the at least one virtual balancing account associated with the respective environmental attribute to the at least one chemical product identifier. One or more environmental attribute(s) may be assigned to the at least one chemical product. Assignment may include converting the one or more balancing unit(s) to one or more environmental attribute(s), wherein the one or more balancing unit(s) are de-allocated from the at least one virtual balancing account associated with the respective environmental attribute.

Assigning at least one environmental attribute to the at least one chemical product may include converting the one or more balancing unit(s) to one or more environmental attributes(s), wherein the one or more balancing unit(s) are deallocated from at least one virtual balancing account associated with the respective environmental attribute. Assigning at least one environmental attribute to the at least one chemical product may include providing a chemical product identifier associated with the produced chemical product and allocating the environmental attribute to the chemical product. Assigning at least one environmental attribute associated with input material(s) to chemical product(s) may include the linking of the chemical product identifier with the environmental attribute. The chemical product identifier may be associated with the physical entity of the chemical product. This way the virtual identifier of a material may be uniquely linked to the physical material. Such linking may include a physical or virtual link of identifiers uniquely associated with the physical material. For physical linking a tag or code may be physically connected to the material, e.g., by printing a QR code on the packaging. For virtual linking different identifiers associated with the physical material may be linked. For example, an order number, a batch number, LOT number or a combination thereof may be linked.

For illustration purposes and to further explain the methods shown in FIGS. 9-11, FIG. 12 shows an example of chemical products produced by the chemical production network from input materials through different intermediates.

The chemical production network may include multiple production chains with one or more intermediate stage. The production chains may have one common starting point as for example provided by the steam cracker. The cracker feedstock may comprise fossil and non-fossil input materials. The fossil input materials may comprise gaseous or liquid hydrocarbon containing feedstock such as naphtha and/or low-pressure gas or ethane. The non-fossil input materials may comprise bio-based and/or recycled feedstock.

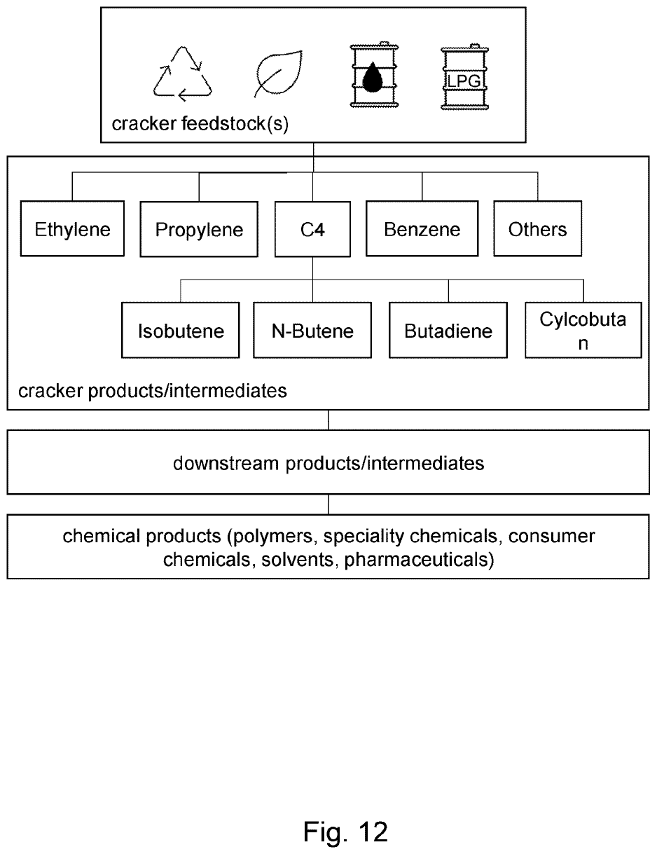

In steam cracking the feedstock is diluted with steam and heated in a furnace to reaction temperatures between 500-1000 °C. After the cracking, the gas is quenched to stop the reaction in a transfer line exchanger. The cracker products produced in the reaction depend on the composition of the feed, on the hydrocarbon to steam ratio and on the cracking temperature and furnace residence time. In the example shown in FIG. 12, cracker products include light alkenes such as ethylene or propylene, C4-hydrocarbons such as Isobutene, N-Butene, Butadiene or Cyclobutane, and/or other hydrocarbons such as aromatic hydrocarbons or mixtures of aromatic hydrocarbons, such as benzene, toluene and/or xylenes (benzene and toluene also abbreviated as BT; benzene, toluene and xylenes also abbreviated as BTX).

The cracker products may be provided to the exit point of the chemical production network. The cracker products may in such embodiment be chemical products. The environmental attributes of the recycled or bio-based input materials may be assigned to such chemical products. The chemical production network may include one or more production chain(s) that further process or chemically convert the cracker intermediates. The cracker intermediates may be further processed or chemically converted by one or more production chain(s) inside the chemical production network. The cracker intermediates may be processed to downstream products. The downstream products may be provided to the exit point of the chemical production network. The downstream products may hence be chemical products. The environmental attributes of the recycled or bio-based input materials may be assigned to such chemical products. The cracker intermediates may be processed via one or more downstream intermediates to chemical products. The environmental attributes of the recycled or bio-based input materials may be assigned to such chemical products. Chemical products may include polymers, specialty chemicals, consumer chemicals, solvents, pharmaceuticals, or the like.

For illustration purposes and to further explain the methods for allocating environmental attributes to virtual balancing accounts and assigning environmental attributes to chemical products as described in the context of FIGS. 9-11, FIG. 13 illustrates examples of the conversion to balancing units and assignment to chemical products.

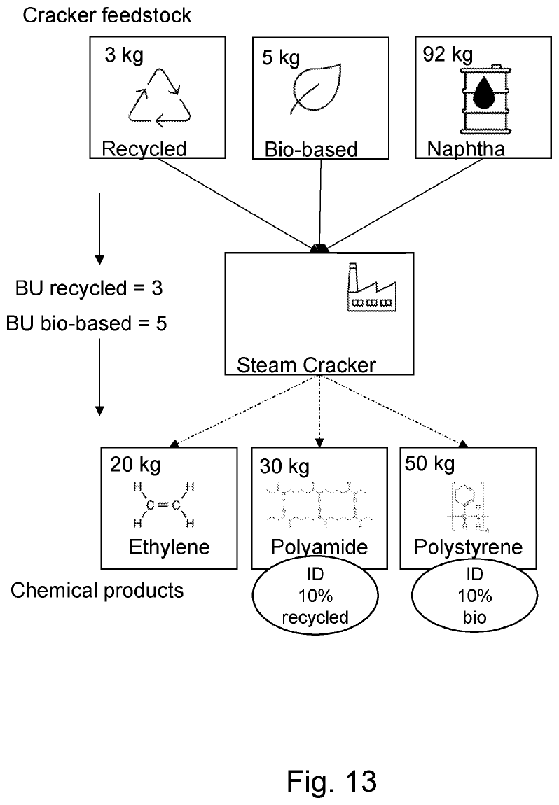

Based on the non-limiting example of FIG. 13 bio-based and recycled feedstock may be provided to the steam cracker. For illustrative purposes the following input materials may be provided to the steam cracker in the following amounts:
3 kg recycled input material such as pyrolysis oil produced from mixed plastics waste,
5 kg bio-based input material such as bio-naphtha from vegetable oil that is kosher and vegan,
92 kg of naphtha.

On providing these input materials to the steam cracker, the input materials enter the chemical production network. The environmental attributes of the recycled and bio-based input materials may be provided to the computer interface configured to convert such attributes to balancing units and to allocate the balancing units to respective virtual balancing accounts. In the simplest example, the balancing units (BUs) may correspond to the amounts of the respective materials. In the example, the environmental attribute pyrolysis oil from recycled mixed plastics waste may correspond to 3 BUs and the environmental attribute bio-naphtha from bio-based food waste may correspond to 5 BUs. In this example a simplified weight-based approach is used for illustrative purposes only. Other approaches may be based on energy, atom counting such as carbon atoms, molecule counting such as methane, or include losses that occur in production.

In more elaborate embodiments the balancing units may be determined based on a more complex conversion factor taking chemical and/or physical differences between input materials and their associated yield into account. The conversion factor may quantify the differences in chemical and/or physical properties of replacing fossil input material(s) by non-fossil input material(s). The conversion factor may relate the use of conventional input material(s) to the use of input material(s) associated with one or more environmental attribute(s). The conversion factor may depend on carbon atoms, methane molecules, energy properties, process properties or any other suitable factors for quantifying the environmental impact of the environmental attribute. For instance, the lower or higher heating value (LHV, HHV) of the fossil and the non-fossil input material may be taken into account. Further for instance, material losses that occur in the processing of the fossil or the non-fossil input material may be taken into account. Further for instance, exempted steam cracker products, intermediates or production chains may be taken into account. Further for instance, only pre-selected production chains may be taken into account. This way the environmental impact of the non-fossil input materials may be quantified with reference to fossil input materials.

The steam cracker may produce cracker products, which may be further processed and chemically converted. In the illustrative example, 20 kg ethylene as cracker product, 30 kg polyamide and 50 kg polystyrene may be provided to the exit point of the chemical production network. Since for the production of such products 3 kg recycled and 5 kg bio-based input materials were used, the environmental attributes allocated in BUs to the respective balancing accounts can be assigned to such products. For instance, 3 BUs recycled may be assigned to Polyamide, which corresponds to 10% recycled content, and 5 BUs bio-based may be assigned to Polystyrene, which corresponds to 10% bio-based content.

For illustration purposes and to further explain the methods for assigning environmental attributes from the balancing account to the chemical product identifier as described in the context of FIGS. 1-13, FIG. 14 a, b illustrate examples of data structures for assigning environmental attributes from the balancing account to the chemical product identifier.

Based on the non-limiting example of FIG. 12 bio-based and recycled feedstock may be provided to the steam cracker, converted to the balancing units of as shown in FIG. 13 and allocated to the balancing accounts. Balancing account recycled has in this example a balance of 3 BUs and balancing account bio-based has a balance of 5 BUs.

For assignment of such BUs to the chemical product, the chemical product identifier may be provided. The chemical product identifier may be associated with the chemical product provided to the exit point of the chemical production network. The chemical identifier may relate to the chemical product specification. The chemical identifier may relate to the chemical product specification and the environmental attribute.

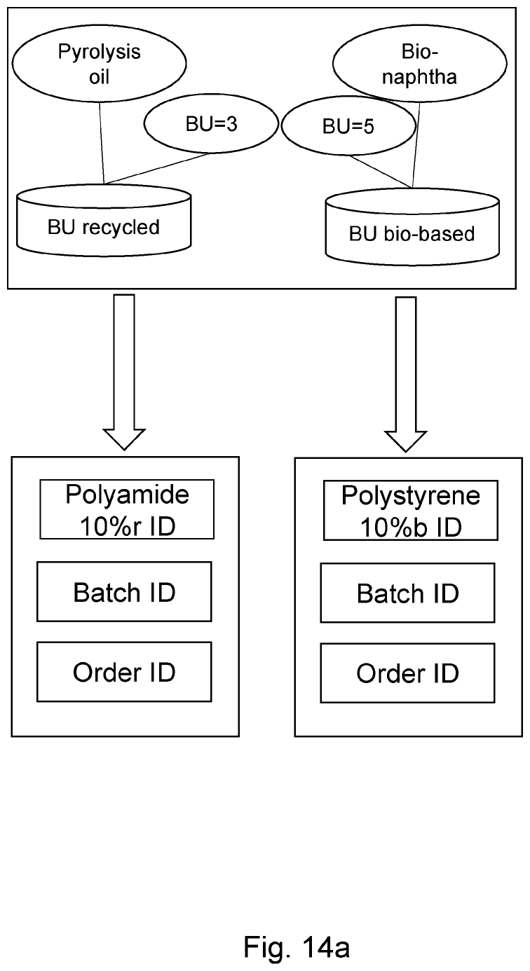

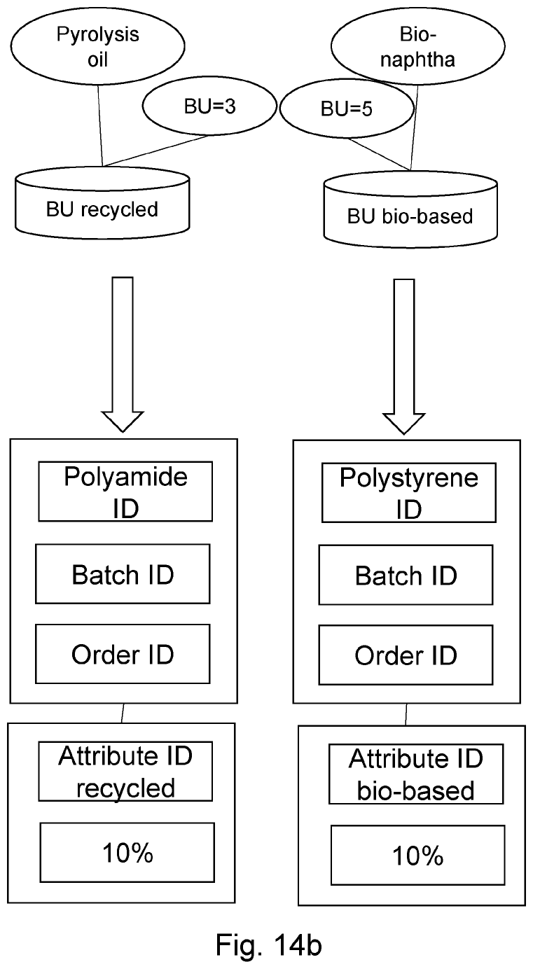

As shown in FIG. 14a, the chemical identifier may relate to the chemical product specification Polyamide or Polystyrene and the environmental attribute 10% recycled or bio-based content. The chemical product identifier may be provided for pre-defined chemical products associated with pre-defined environmental attribute(s). In this embodiment, the number of BUs required for the respective chemical products is pre-defined and a further conversion of BUs to respective environmental attribute(s) is not required. This way the management of input materials and chemical products with environmental attributes is less dynamic and can be simplified.

As shown in FIG. 14b, in addition to the chemical identifier an environmental attribute identifier may be provided. The chemical identifier may relate to the chemical product specification Polyamide or Polystyrene. The chemical product identifier may be provided for pre-defined chemical products. The environmental attribute identifier may relate to the environmental attribute 10% recycled or bio-based content. The environmental attribute identifier may be linked to the chemical product identifier. The environmental attribute identifier may be provided for pre-defined environmental attribute types. For assignment, the BUs may be assigned to the environmental attribute identifier. For assignment, the BUs may be converted to the environmental attribute and the environmental attribute such as 10% recycled content may be assigned to the environmental attribute identifier. In this embodiment, the number of BUs required for the respective chemical products is not pre-defined and can be flexibly assigned. This way chemical products with environmental attributes tailored to customer needs can be provided.

The chemical product identifier may be uniquely linked to the physical entity of the chemical product. In one embodiment, the batch identifier and the order identifier may be provided and/or linked to chemical product identifier. This way the chemical product identifier may be uniquely linked to the physical entity of the chemical product exiting the chemical production network. In other embodiments the chemical identifier may be linked to the physical entity of the chemical product by way of a physical identifier with encoded chemical product identifier and physically connected to the chemical product. For example, a tag or a QR code may be physically connected to the chemical product and the chemical product identifier may be encoded into the tag or QR code. This way the chemical product identifier may be uniquely linked to the physical entity of the chemical product exiting the chemical production network.

For illustration purposes and to further explain the methods for allocating environmental attributes to virtual balancing accounts as described in the context of FIGS. 9-14, FIGS. 15a-c illustrate examples of balancing accounts to manage the allocation and assignment of balancing units.

Based on the non-limiting example of FIG. 13 bio-based and recycled feedstock may be provided to the steam cracker. Material data related to the bio-based and recycled feedstock and the respective environmental attributes may be provided to the computing interface configured to convert such attributes to balancing units and to allocate the balancing units to respective virtual balancing accounts. In the simplest example, the environmental attribute pyrolysis oil from recycled mixed plastics waste may correspond to 3 BUs and the environmental attribute bio-naphtha from bio-based vegetable oil may correspond to 5 BUs. The so determined BUs may further be associated with metadata signifying the respective environmental attributes as recycled, pyrolysis oil and recycled tires as well as bio-based, bio-naphtha, vegetable oil.

For allocation the virtual balancing system may be provided. The virtual balancing system may include balancing accounts associated with metadata relating to environmental attribute, input material type, waste stream type, biomass type, and balancing units. As for example shown in FIG. 15a, the balancing accounts may be associated with metadata relating to environmental attribute type recycled or bio-based, input material type pyrolysis oil, bio-naphtha or bio-gas, input material origins tires, mixed plastics waste, vegetable oil or food waste, respectively. Furthermore, the balancing accounts may be associated with an account balance quantifying the BUs.

For allocation of the 3 BUs and the 5 BUs the metadata of the balancing accounts may be matched with the metadata associated with such BUs. The BU metadata may be provided from the input material data. Once a match in metadata is found the respective BUs are allocated to the matched account. Hence in the example, the 3 BUs pyrolysis oil from recycled mixed plastics waste may be allocated to account BU recycled 2 and the 5 BUs bio-naphtha from bio-based vegetable oil may be allocated to account BU bio 1.

The virtual balancing system may be based on balancing accounts with static or dynamic metadata. For instance, if metadata provided via the environmental attribute associated with the input material does not correspond to any metadata of balancing accounts provided, a new account associated with such metadata may be provided. Alternatively, or additionally, the environmental attribute may be allocated to the account associated with the greatest match in metadata. Here greatest may refer to the maximal number of matching metadata points, in particular account metadata points matching at least in part with environmental attribute metadata points. For example, the environmental attribute may provide more metadata than any balancing account. In such a scenario the account with metadata points matching at least in part with metadata points of the environmental attribute may be selected for allocation.

The metadata structure shown in FIG. 15a is only one example. FIG. 15b illustrates another example. Here a hierarchical tree of metadata (leafs) with different levels may be associated to the balancing accounts. For example, the top level may be bio-based or recycled, the next level may specify the input material type for recycled or bio-based, respectively, and so on.

The metadata structures shown in FIG. 15a, b are only non-limiting examples. FIG. 15c illustrates another example. In this example the virtual accounting system may be associated with metadata relating to the environmental attribute recycled and the input material type pyrolysis oil. The virtual accounting system may include two balancing accounts, BU1 and BU2. BU1 may be associated with waste stream type tires and the balancing units allocated to BU1. BU2 may be associated with waste stream type mixed plastics and the balancing units allocated to BU2.

FIG. 15a-c are only examples based on metadata attached to the virtual accounting system or the balancing accounts. Other options to realize such a data structure exist. For example, the virtual balancing system and the balancing accounts may be fully abstracted and each BU entering the system may have its own metadata.

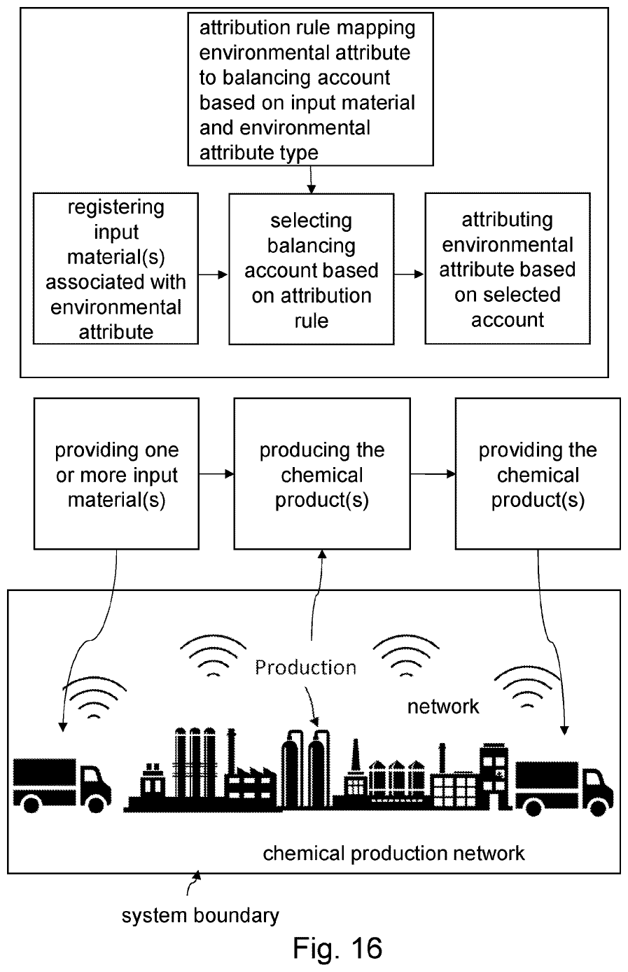

FIG. 16 illustrate an example of a method for attributing or allocating at least one environmental attribute to a balancing account based on an attribution rule. The input materials such as pyrolysis oil, bio-naphta or bio-gas may be provided to the chemical production network. The input materials may enter the system boundary of the chemical production network at the entry point, such as a stream cracker. The input materials may be used in the chemical production network to produce one or more chemical products(s) from the input materials. Chemical products(s) may be MDI, TDI, PA6, EPS, PC, Polyols, Caprolactam, adipic acid, HMD, Polyamides.

On entry of the input material, input material data may be provided via a network to the interface of the operating system. The input material identifier associated with the respective input material, the environmental attribute associated with the respective input material, a value associated with the input material and the quantity of input material provided to the chemical production network may be provided. Such data may be provided via a network on entry of the system boundary, or the data may be transferred from a computing system to the operating system.

The input material identifier may be associated with the physical entity of the input material entering the chemical production network. A tag may be placed on the delivery truck providing the input material identifier and related data. The input material identifier may be a LOT number assigned to a certain quantity or group of input material(s) and/or the material identifier relates to an order number assigned to the transfer of a certain quantity or group of input material(s) for the chemical production network. The data related to the input material identifier may include the environmental attribute associated with the respective input material, a value associated with the input material, the quantity of input material and the certificate certifying the environmental attribute. The quantity of input material may be a measured quantity of input material fed to a plant or storage of the chemical production network for producing one or more chemical product(s) from the input material(s).

The input material identifier may be provided and the related data may be accessed via a network and retrieved from a input product system. The at least one environmental attribute, the value associated with the input material, and the quantity of input material provided to the chemical production network be may be associated with the input material identifier and authentication information. The providing of the input material to the chemical production network may trigger generation of a transaction record assigning or attributing the input material identifier from an input material producer identifier to an input material user identifier.

The environmental attribute may be allocated or attributed to an input material account associated with the respective environmental attribute type. For allocation or attribution, attribution rules mapping the environmental attribute to the input material account may be provided. The attribution rules may depend on the input material type and the environmental attribute type. The attribution rule may be linked to meta data relating to input material type and environmental attribute type.

Similarly, the input material account may be linked to meta data relating to input material type and environmental attribute type. Based on the input material type and the environmental attribute type the input material account the respective metadata may be matched and the input material account may be selected.

For the attribution or allocation, environmental units may be determined, wherein the environmental units may be equivalents in mass, in heating value, in methane, in carbon atoms or the like. For example, the quantity of input material such as pyrolysis oil may be converted to environmental units. This may include a conversion factor that takes account of the chemical difference between fossil-based input materials such as naphtha and methane and non-fossil-based input materials such as bio-naphtha or pyrolysis oil. In one example the conversion factor may relate to the lower heating value of the pyrolysis oil in relation the lower heating value of naphtha or methane. The conversion factor may for instance include the ratio of the lower heating value of pyrolysis oil and naphtha or methane. This way the chemical difference between the fossil and the recycled input material can be considered.

The environmental units may be attributed or allocated to the selected input material account. For example, the attribution rule may be associated with account for pyrolysis oil. On production of the output products corresponding environmental units may be assigned to the chemical product(s). An attribution rule may specify assignment of environmental units to the chemical product(s).

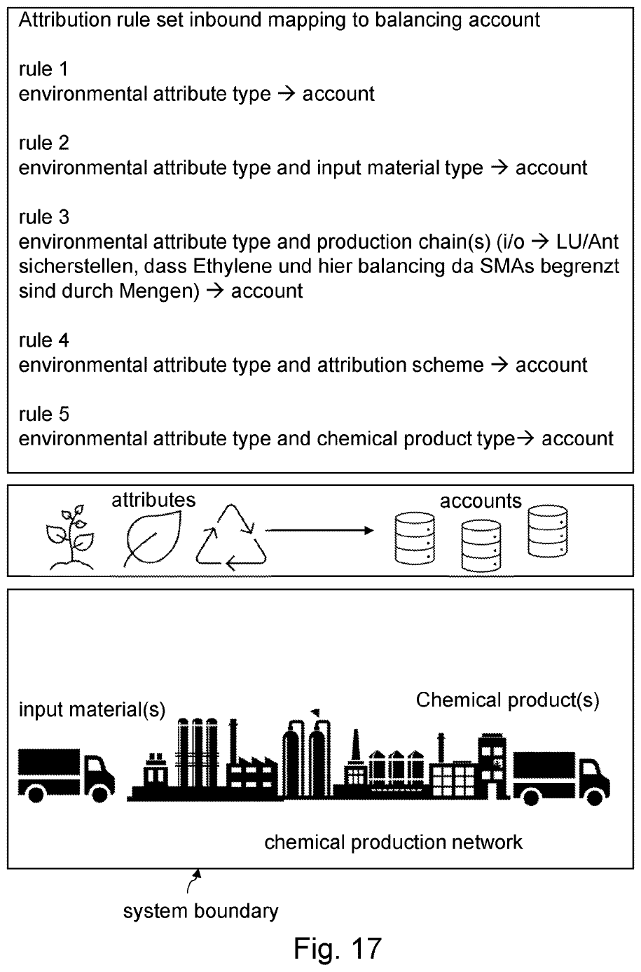

FIG. 17 illustrate examples of attribution rules for attributing at least one environmental allocate attribute to a balancing account.

Possible inbound attribution rules 1 to 5 mapping environmental attributes to input material accounts are illustrated in FIG. 17. The attribution rule may depend on the environmental attribute type such as recycled input material. The attribution rule may depend on the environmental attribute type such as recycled input material and the input material type such as recycled input material and pyrolysis oil based on plastics waste. The attribution rule may depend on the environmental attribute type such as recycled input material and production chain such as the ethanol production chain. The attribution rule may depend on the environmental attribute type such as recycled input material and the attribution scheme such as mass balance with and without free attribution. The attribution rule may depend on the environmental attribute type such as recycled input material and the chemical product type such as polyurethane.

Based on such attribution rules the environmental attributes registered on entry to the system boundary may be attributed or allocated to respective input material accounts. Environmental attribute types may include bio-based, recycled, renewable or the like.

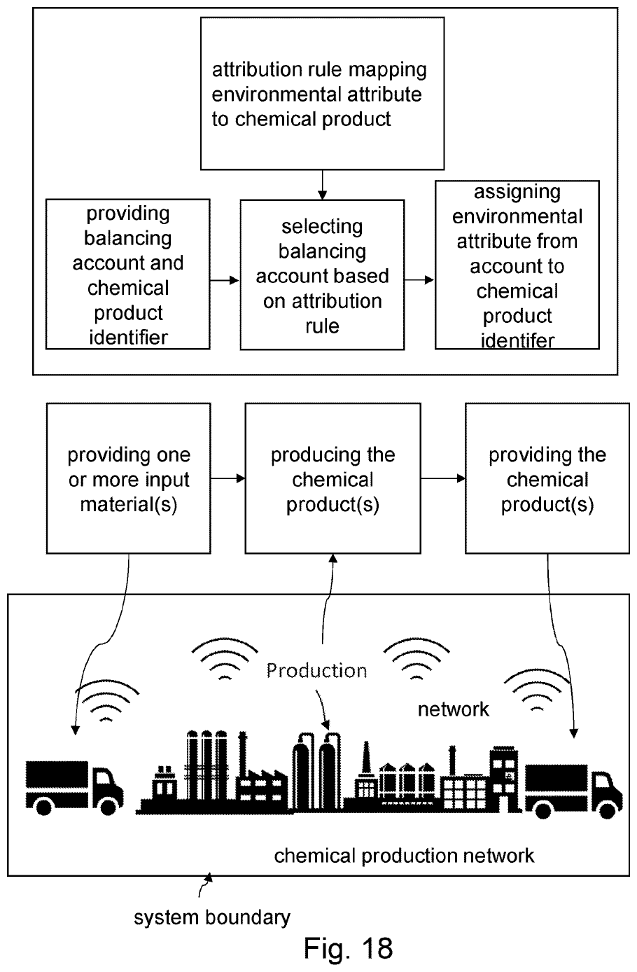

FIG. 18 illustrate an example of a method for assigning or attributing at least one environmental attribute to a chemical product id based on an attribution rule.

Similar to FIG. 17 the input materials are provided to the chemical production network and chemical products are produced by the chemical production network. On registration of the input materials environmental attributes associated with the input materials are attributed to the input material accounts as described herein and in the context of FIG. 8. The input material accounts, the chemical product identifier associated with the chemical product and the chemical product environmental attribute may be provided. The chemical product attribute may comprise one or more environmental attribute type(s). The chemical product attribute may comprise different numbers of environmental unit(s) for respective environmental attribute type(s).

Based on the chemical product identifier and the target environmental attribute the attribution rule may be provided. The attribution rule may map the environmental attribute type to the chemical product type. For example, the attribution rule may include metadata related to the environmental attribute type and to the chemical product type. Similarly, the input material account may include metadata related to the environmental attribute type and to the chemical product type. Based on the matching of metadata the input material account may be selected. This way the account for attributing environmental attributes from account to the chemical product identifier may be determined.

The respective environmental attributes from the input material account may be assigned to the chemical product. On production of the chemical product corresponding environmental units may be assigned to the chemical product.

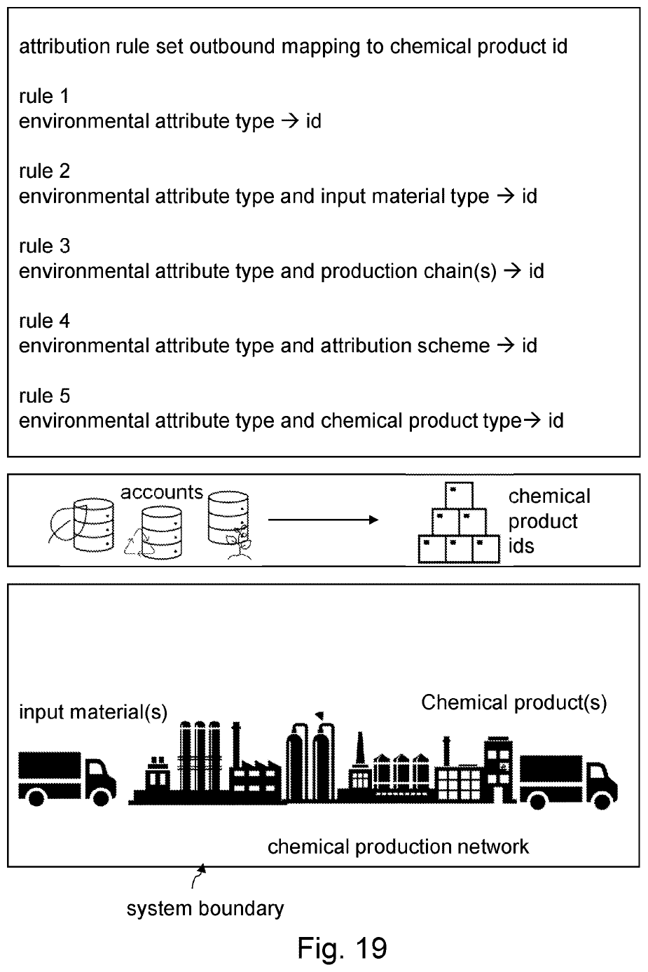

FIG. 19 illustrate examples of attribution rules for assigning or attributing at least one environmental attribute to a chemical product id based on an attribution rule.

Possible outbound attribution rules 1 to 5 mapping environmental attributes from input material accounts to chemical product are illustrated in FIG. 19. The attribution rule may depend on the environmental attribute type such as recycled input material. The attribution rule may depend on the environmental attribute type such as recycled input material and the input material type such as recycled input material and pyrolysis oil based on plastics waste. The attribution rule may depend on the environmental attribute type such as recycled input material and production chain such as the ethanol production chain. The attribution rule may depend on the environmental attribute type such as recycled input material and the attribution scheme such as mass balance with and without free attribution. The attribution rule may depend on the environmental attribute type such as recycled input material and the chemical product type such as polyurethane.

Based on such attribution rules the environmental attributes registered on entry to the system boundary and provided by the input material accounts may be assigned to respective chemical products. Environmental attribute types may include bio-based, recycled, renewable or the like.

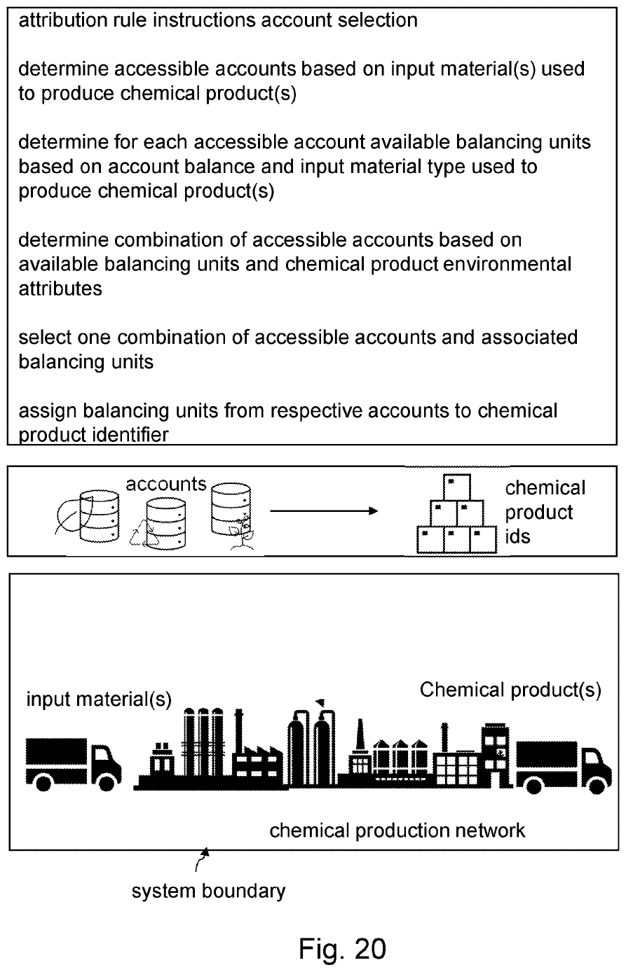

FIG. 20 illustrate examples of attribution rule instructions for selecting at least one account. Similar to FIGS. 17 and 18, the input materials are provided to the chemical production network and chemical products are produced by the chemical production network. On registration of the input materials environmental attributes associated with the input materials are attributed to the input material accounts as described herein and in the context of FIG. 18.

FIG. 20 illustrates attribution rule instructions configured to select an account. Depending on the chemical product and the input materials such chemical product is produced from different input material accounts may be accessible for the chemical product. The input materials may be determined from a bill of materials including the recipe for the production chain up to the chemical product. The production chain may include the input materials that enter the system boundary of the chemical production network at any stage. From the input materials used to produce the chemical product the accessible accounts associated with such input material types may be determined.

For each accessible account accessible environmental units may be determined from the account balance and the input material type used to produce chemical product. Such determination may result in one or more accounts being accessible for the chemical product and the target environmental input. For example, the target environmental attribute may refer to pyrolysis oil irrespective of the waste stream. The input material account for pyrolysis oil from different waste streams may hence be accessible accounts. The depending on the respective account balance one or more combinations of accounts may fulfil the input material account. One combination of accessible accounts may be selected for example based on the combination with the highest account balance in respective accounts. This way the environmental attributes required by other stricter target environmental attributes may still be fulfillable.

On providing the chemical product, the environmental units from the respective account(s) may be assigned to the chemical product identifier. This way the chemical product can be uniquely associated with the target environmental attribute via the chemical product identifier.

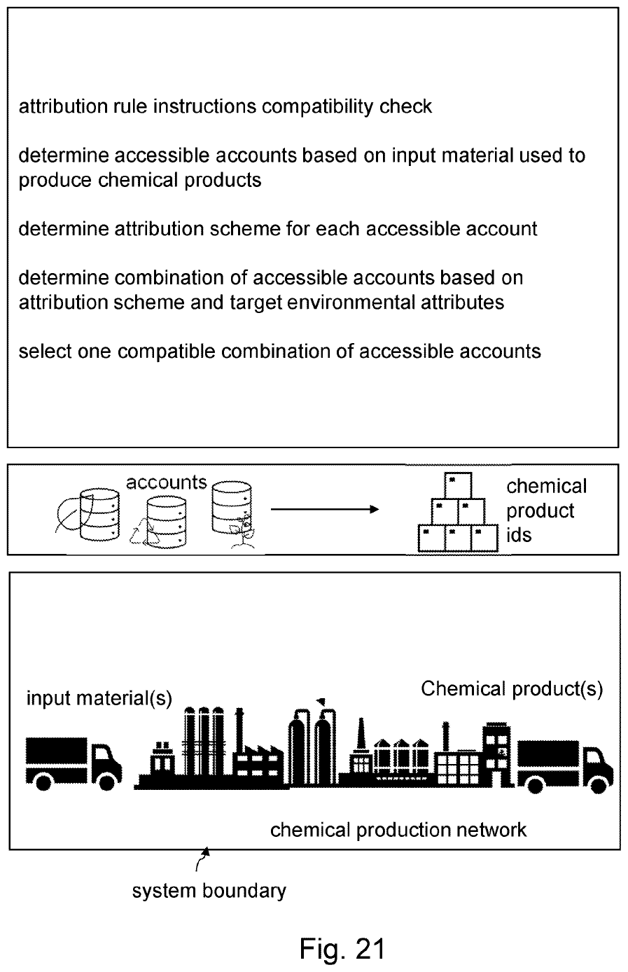

FIG. 21 illustrate examples of attribution rule instructions for the compatibility check of accounts.

Similar to FIGS. 17-19, the input materials are provided to the chemical production network and chemical products are produced by the chemical production network. On registration of the input materials environmental attributes associated with the input materials are attributed to the input material accounts as described herein and in the context of FIG. 18.

FIG. 21 illustrates attribution rule instructions configured to check compatibility between attribution schemes. Depending on the accounts accessible to accommodate the target environmental attribute different attribution schemes may apply. For instance, one account may be associated with a book and claim scheme, while another account may be associated with a segregated scheme. Further for instance, one account may be associated with a mass balance scheme, while another account may be associated with a segregated scheme. Further for instance, one account may be associated with a mass balance scheme with free attribution, while another account may be associated with a mass balance scheme without free attribution. The different attribution schemes may be mutually exclusive. The different attribution schemes may be compatible with each other in the sense that environmental units from a first account associated with a first attribution scheme may be combined with environmental units from a second account associated with a second attribution schemes and vice versa. The different attribution schemes may be compatible with each other in the sense that environmental units from first account associated with a first attribution scheme may only be combined with environmental units from a second account associated with a second attribution schemes. A reverse combination may be excluded. Similarly, the attribution rule associated with the target environmental attribute may be compatible or not compatible with the accounts. Compatibility rules specifying the compatibility of different attribution schemes associated with respective accounts may be provided from a data base. Compatibility rules specifying the compatibility of different attribution schemes associated with respective accounts may relate to accounts and/or the target environmental attribute. The target environmental attribute and/or the accounts may include respective metadata specifying the attribution scheme. Depending on such compatibility rules the compatible combination of accessible accounts and/or target environmental attribute may be determined by matching the metadata. This way it can be ensured that the target environmental attribute includes only compatible environmental attributes.

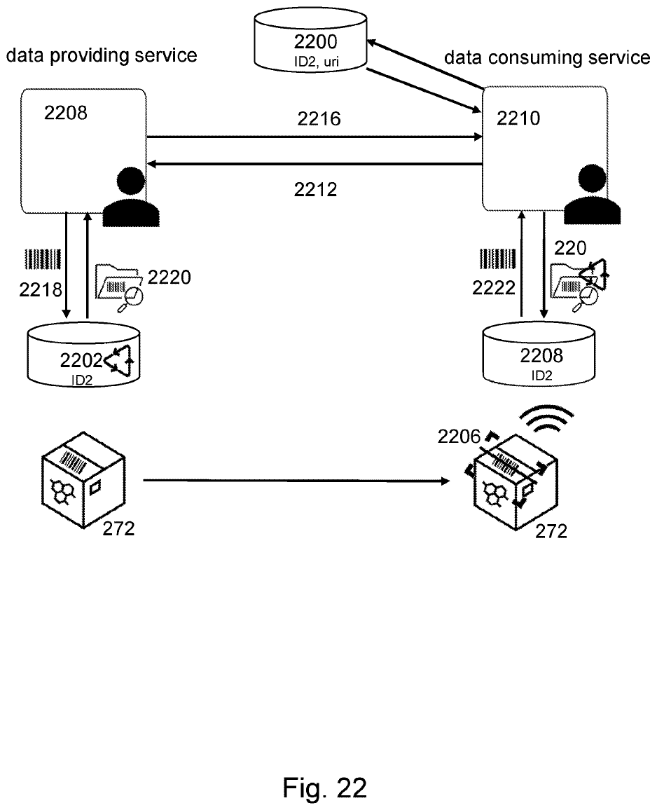

FIG. 22 illustrates schematically an example of a method or apparatus for providing environmental attributes associated with chemical products to a data consumer (e.g., a customer) via a decentral network.

The chemical product 272 as produced by the chemical production network 110 may be provided in association with a digital asset such as an identifier (e.g., including a chemical product identifier and environmental attribute identifiers) as described in the context of FIGS. 14a and 14b. The digital asset may include the chemical product identifier. The digital asset may include one or more environmental attribute(s) such as the product carbon footprint, recycled content or bio-based content. The digital asset may relate to one or more environmental attribute(s) such as the product carbon footprint, recycled content or bio-based content. The digital asset may include a digital representation of one or more environmental attribute(s) such as the product carbon footprint, recycled content or bio-based content.

The digital asset may further include or relate to authentication and/or authorization information linked to the chemical product identifier. The authentication and/or authorization information may be provided for authentication and/or authorization of a data providing service 2208 and/or data consuming service 2210. The chemical product identifier may include or relate to a decentral identifier, that is uniquely associated with the chemical product. The decentral identifier may be connected to the digital representation of the environmental attributes. The digital representation may include a representation for accessing the environmental attributes or parts thereof. The decentral identifier may include a Universally Unique IDentifier (UUID) or a Digital IDentifier (DID). The decentral identifier may include any unique identifier uniquely associated with a data owner and/or chemical product. The data owner may be the producer of the chemical product. Via the decentral identifier and its unique association with the data owner and/or chemical product, access to the material configuration data may be controlled by the data owner.

The digital asset including the digital representation of one or more environmental attribute(s) such as the product carbon footprint, recycled content or bio-based content may be stored in a decentral data base 2200. The one or more environmental attribute(s) such as the product carbon footprint, recycled content or bio-based content may be stored in a data base 2202 associated with the data owner, such as the producer of the chemical product 272.

The chemical product 272 may be physically delivered to a customer (or other user of the chemical product). The chemical product may be connected with a QR-code having encoded the chemical product identifier. The user of the chemical product may read the QR-code through a QR-code reader 2206. The chemical product identifier may be provided to a data base 2208 associated with the user or customer of the chemical product 104. In other embodiments the user or customer of the chemical product may retrieve the chemical product identifier through the decentral data base 2200.

The data owner in this example may be the chemical product producer, the chemical product customer/user, or the end product producer. The data owner may comprise any entity generating data. The data generating node may be coupled to the data owner or the entity owning or producing physical products from or for which data is generated. The data may be generated by a third-party entity on behalf of the entity owning physical products from or for which data is generated.

The data consuming service 2210 may comprise computer-executable instructions for accessing and/or processing data, such as chemical product data, associated with the data owner. The data providing service 2208 may comprise computer-executable instructions for providing and/or processing data, such as plastic additive data, associated with the data owner for accessing and/or processing by the data consuming service 2210.

Based on the received chemical product identifier a request to access the environmental attributes associated with the chemical product identifier may be triggered by the data consuming service 2210 as signified by arrow 2212. The chemical product identifier may be provided to the data providing service 2208 associated with or of the producer of the chemical product 272. In addition, authentication and/or authorization information may be provided.

The request may be authenticated and/or authorized to access the environmental attributes associated with the chemical product identifier. Based on successful authorization and/or authentication access to the environmental attributes associated with the chemical product identifier may be granted.

For access the chemical product identifier may be provided to the data providing service 2208 as signified by arrow 2212. The data providing service 2208 may use the received chemical product identifier to retrieve the environmental attributes associated with the chemical product 272 as signified by arrows 2218 and 2220. The environmental attributes associated with the chemical product 272 provided to the data providing service 2208 may be provided to the data consuming service 2210 as signified by arrow 2216. The environmental attributes associated with the chemical product 272 may be stored in the data base 2208 associated with the user/purchaser of chemical product 272 as signified by arrow 2222.

Through the output identifier or decentral identifier the environmental attributes can be uniquely associated with the chemical product. Through the decentral network the environmental attributes may be transferred between the producer of the chemical product and the user/customer of the chemical product. This way the environmental attributes can be shared with unique association to the chemical product and without central intermediary directly between the value chain players. This allows for transparency of environmental attributes across the value chain and positive environmental impacts from chemical products produced by the chemical production network 110 can be tracked through the value chain.

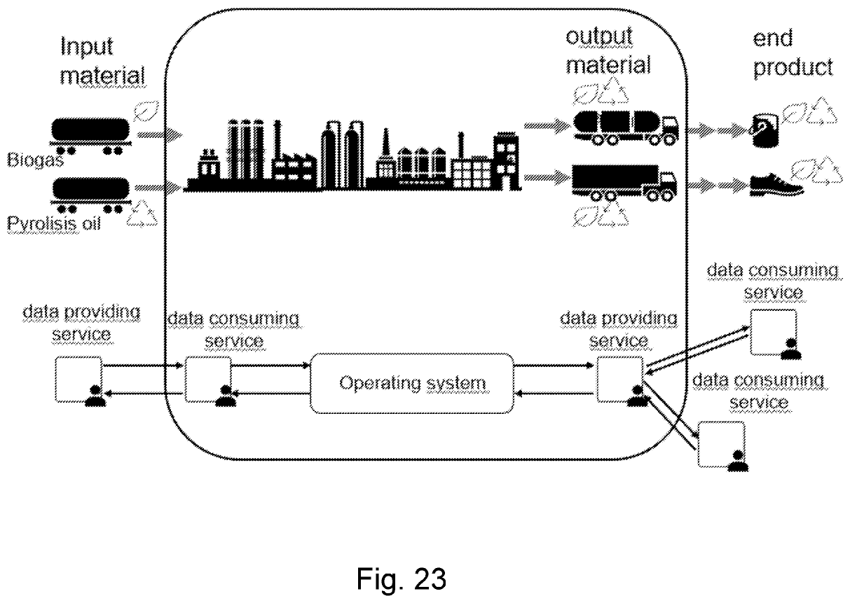

FIG. 23 illustrates schematically an example of a method or apparatus for providing environmental attributes associated with chemical products across value chains via the decentral network.

In the example of FIG. 23 a fully connected value chain including the chemical production network is illustrated. In the example, the input material provider, the chemical product producer, the chemical product user/customer and the end product producer may be connected to the decentral network as described in the context of FIG. 22. Environmental attributes may be provided via the ID based schema described in the context of FIGS. 2-21 in the form of digital assets or chemical product passports associated with the physical entity of the input material, the chemical product, any intermediate product or the end product.

The input material provider may provide the input materials such as bio-gas or pyrolysis oil. The environmental attributes of the input material may be provided through the data providing service connected to the decentral network as described in the context of FIG. 22. The chemical product producer may produce the chemical product from the input material(s) provided to the chemical production network. The chemical product producer may access the environmental attributes associated with the input material through a data consuming service connected to the decentral network as described in the context of FIG. 22. The chemical product producer may manage the environmental attributes via the production operating system as described in the context of FIGS. 1 to 4. The chemical product producer may assign the environmental attributes associated with the input materials or environmental attributes associated with the chemical production network such as the carbon footprint, to the chemical product s as described in the context of FIGS. 1 to 4. The chemical product producer may provide the environmental attributes associated with the chemical product through the data providing service connected to the decentral network as described in the context of FIG. 22. The chemical product user/customer or the end product producer may access the environmental attributes associated with the chemical product through the data consuming service connected to the decentral network as described in the context of FIG. 22.

The respective data owners in this example may be the input material producer, the chemical product producer, the chemical product user/customer, the end product producer. The data owner may comprise any entity generating data. The data generating node may be coupled to the data owner or the entity owning or producing physical products from or for which data is generated. The data may be generated by a third-party entity on behalf of the entity owning physical products from or for which data is generated.

The data consuming service may comprise computer-executable instructions for accessing and/or processing data, such as chemical product data, associated with the data owner. The data providing service may comprise computer-executable instructions for providing and/or processing data, such as plastic additive data, associated with the data owner for accessing and/or processing by the data consuming service.

In the example of FIG. 23 the decentral identifier may relate to the end product. Such decentral identifier may be provided to the value chain participants. Via the end product specific decentral identifier data associated with the end product produced from the chemical product may be gathered across the production chain and assigned to the end product specific decentral identifier. For example, the one or more environmental attribute(s) associated with the end product may be derived from the environmental attribute(s) associated with the chemical product, the input material or any other product entity present in the value chain of the end product.

This way the environmental attributes of input materials, chemical products and any products produced from chemical products may be tracked through the value chain up to the end product. By tracking the environmental attributes of materials in such way the information can be made transparent across the value chain while the information flow can be controlled by the participants in the supply chain. In addition, the environmental attributes can be handled according to the individual participants needs by production operating systems as described in the context of FIGS. 1 to 4.

Overall, such tracking enables tracking of positive environmental impact by individual supply chain participants, which makes positive environmental impacts transparent and attributable to individual supply chain participants.

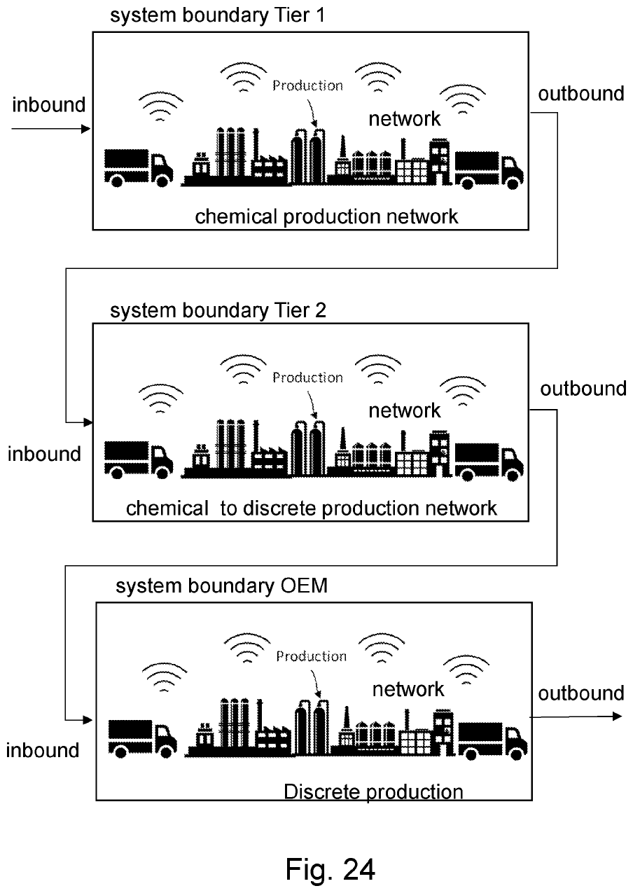

FIG. 24 illustrates an example of a method for producing at least one chemical product associated with at least one environmental attribute for a supply chain.

The example illustrates two tiers and an original equipment manufacturer. Tier 1 may be a chemical producer operating the chemical production network. The chemical production network may be associated with a system boundary. The system boundary may signify the physical boundary of the chemical production network. The input material(s) entering the chemical production network at any stage of the production network, or the system boundary of the chemical production network may signify entry points to the chemical production network. On entry of input material(s) associated with one or more environmental attributes the environmental attribute(s) may be decoupled from the physical material flow of the input material(s) through the chemical production network. Such separation of the physical input material and the virtual environmental attribute may be provided by the method for registering at least one input material associated with at least one environmental attribute as described herein and as for example illustrated in FIG. 8a (inbound). Chemical product(s) may be produced from input material(s) by the chemical production network. At least one chemical product may be produced by the chemical production network and the chemical product may be linked to at least one environmental attribute as described herein and illustrated in FIG. 8b (outbound).

The chemical product(s) associated with at least one environmental attribute as produced by the chemical production network may be provided to the next tier. In the illustrated example tier 2 may be a production network producing discrete products from chemical product(s) or chemical products. Discrete products may be any products associated with distinct physical units. Discrete manufacturing in contrast to process manufacturing uses such discrete products to assemble other discrete products. Chemical production in contrast uses process manufacturing where input material(s) are mixed and chemically converted to chemical output product(s). Such output product(s) are chemical product(s) that may be transferred in discrete units, such as containers or wreath. The chemical product(s) are such are, however, not discrete product(s). The chemical to discrete production network may be associated with a system boundary as described above. Similar to the chemical production network, the chemical to discrete production network may on inbound register input material(s) associated with at least one environmental attribute, which may be the chemical product(s) of the chemical production network as described herein or illustrated in FIG. 8a (inbound). Discrete output product(s) may be produced by the chemical to discrete production network. At least one discrete output product may be linked to at least one environmental attribute (outbound).

The discrete product(s) associated with at least one environmental attribute as produced by the chemical to discrete production network may be provided to an original equipment manufacturer producing end products. As described herein and in the context of FIGS. 8a and 9, the discrete product(s) and associated environmental attribute(s) may be decoupled on registration (inbound). As described herein and illustrated in FIGS. 8b and 10 after decoupling the environmental attribute may be linked to the end product as described herein and illustrated in FIG. 8b (outbound).

The one or more environmental attribute(s) assigned to the chemical product identifier may be used to provide at least one discrete product identifier relating at least one discrete product or at least one end product identifier specifying at least one end product of a product supply chain to the one or more environmental attribute(s). The chemical product associated with one or more environmental attribute(s) may be used to produce at least one discrete product or at least one end product of a product supply chain associated with the one or more environmental attribute(s).

This way the environmental attributes associated with any input material(s) may be tracked through the value chain up to the end product. The methods, apparatuses and systems described herein enable transparency from early stages of the value chain on chemical product level to end stage of the value chain on end product level. Through linking of physical material and environmental attribute environmentally friendly products and more sustainable production can be made transparent and tracked.

The present disclosure comprises the step (a) providing input material data associated with the input material to an operating system of the chemical production network. As described above with reference to FIGS. 1-2 and 8a, on entry of the input material, the corresponding input material data may be provided via a network to the interface of the operating system. A data provider may be configured to provide material data related to the one or more input material(s) and respective environmental attributes to a computing interface configured to allocate the environmental attributes. The material data may be provided on, prior to or after providing the one or more input material(s) at entry points to the chemical production network. The input material identifier may be associated with the physical entity of the input material entering the chemical production network.

The present disclosure comprises the step (b) determining environmental attributes associated with the input material via a virtual production process. For example, with reference to FIGS. 2 and 3, operating system 120 may initiate virtual production process 300 when it receives input material data for sustainable input material(s) (e.g., 222-224). Using input material data 222-224, virtual production process 300 may parse the input material data and apply a corresponding recipe to determine the environmental attributes associated with the input material(s).

The virtual production process for environmental attributes associated with the input material may also determine the amount of the input material. The determination may be based on a bill of materials, a sales receipt, a recipe and/or any of a wide range of digital documents or files (e.g., input material data) associated with receipt of input material(s). An operating system may parse the input material data to determine the amount of input material that was received. The “amount” of the input material may refer to the volume, amount of substance, and/or mass of the input material. The virtual production process for environmental attributes associated with the input material may also determine a value associated with the input material. For example, an operating system may compute the difference in cost between a sustainable input material and the corresponding equivalent fossil input material to determine the value associated with the input material. The value may be based on average price, actual price, market price or other suitable values to determine the cost of the equivalent amount of fossil input materials. The operating system may store and track the amounts and values corresponding to sustainable input materials. For example, the values may be stored in digital inventories associated with environmental attributes or balancing units.

The present disclosure comprises the step (c) allocating the environmental attributes associated with the input material to a virtual balancing account, wherein the virtual balancing account includes at least one attribution rule for attributing the environmental attributes associated with the input material to a chemical product. The virtual balancing account (or digital inventory) may refer to a digital storage structure that stores data related to environmental attributes. The account may be associated with metadata identifying the account for balancing environmental attributes. The account may be associated with metadata identifying the environmental attributes and the environmental or balancing units allocated to the account. Referring to FIG. 2, operating system 120 may parse input material data 222-224 and allocate the corresponding environmental attributes to virtual balancing accounts 234-236. The account may be associated with metadata identifying the production chain the account is associated with. The account may be associated with metadata identifying the input or chemical product the account is associated with. The account may be part of a balancing system including multiple accounts. The account may hold environmental attributes for transaction.

Environmental attributes may be allocated, added, deleted, withdrawn, or deducted from the account. The virtual balancing account may be associated with environmental attribute types such as recycled or renewable. The virtual balancing account may by associated with input material types such as pyrolysis oil, bio-naphtha, bio-methane, bio-gas or combinations thereof. The virtual balancing account associated with the environmental attribute type recycled may be further associated with waste-stream type such as mixed plastics waste, specific end product waste, e.g., tiers waste or foam waste, post-consumer waste, pre-consumer waste or combinations thereof. The virtual balancing account may be associated with an allocation scheme such as segregated allocation, non-segregated allocation like book and claim, mass balance with free attribution, mass balance without free attribution or combinations thereof.

The present disclosure comprises the step (d) providing a chemical product identifier associated with the chemical product and at least one target environmental attribute. Referring to FIGS. 1-2, operating system 120 may provide a chemical product identifier for one or more of chemical products 262-264. The chemical product identifier may include one or more identifier(s) relating to the chemical product. The identifier may relate to a chemical product class, a specific chemical product and/or properties of the chemical product such as environmental properties. The identifier may include a unique number uniquely associated with the chemical product class, the specific chemical product and/or the properties of the chemical product. The identifier may include one or more specific identifier(s), such as chemical product class identifier, specific chemical product identifier and/or property of the chemical product identifier. Such specific identifier(s) may be uniquely linked to the chemical product. For example, one or more property identifier(s) may be uniquely linked to the chemical product identifier. The chemical product identifier may be uniquely linked to the specific chemical product. This way the chemical product can be uniquely linked to a digital twin of the chemical product specifying specific properties of the chemical product.

The present disclosure comprises the step (e) based on the chemical product identifier and the target environmental attribute, selecting at least one attribution rule and step (f) determining via the at least one attribution rule at least one account for attributing one or more environmental attribute(s) from the at least one account to the chemical product identifier. Referring to FIGS. 1-2, operating system 120 may select at least one attribution rule based, at least in part, on the chemical product identifier and the target environmental attribute. The attribution rule may map the environmental attribute type to the chemical product type. For example, the attribution rule may include metadata related to the environmental attribute type and to the chemical product type. Similarly, the input material account may include metadata related to the environmental attribute type and to the chemical product type. Based on the matching of metadata the input material account may be selected. This way the account for attributing environmental attributes from account to the chemical product identifier may be determined.

The present disclosure comprises the step (g) assigning or attributing the one or more environmental attribute(s) to the chemical product identifier. As for example illustrated in FIG. 1, the chemical production network may be operated by the operating system configured to register inbound environmental attributes, to assign outbound environmental attributes and/or to manage inbound registration as well as outbound assignment via allocation rules. The allocation rules may include allocation schemes as illustrated in FIGS. 6a-c. The chemical production network may include the network as described in the context of FIG. 5 or 7.

The chemical product may be produced from input material that entered the chemical production network. Chemical product data including the chemical product identifier may be provided. The chemical product data may further include input material data associated with the input materials used to produce the chemical product(s), process data associated with the production chain for producing the chemical products and/or data related to the chemical product data, such as a chemical product specification or a chemical product amount.

Based on the input material data environmental attributes associated with the input material(s) used to produce the chemical product(s) may be determined. The input material data may specify the total amount of input material(s) that entered the production chain for producing the amount of chemical product. The input material data may further specify the environmental attributes available for the respective input material(s). From the total amount of input material(s), the number of balancing units for respective environmental attributes available for such input material(s) may be determined. This way the maximum number of balancing units for the respective environmental attribute attributable to the chemical product may be determined.

Based on the environmental attribute(s) attributable to the chemical product, the number of balancing units corresponding to the determined environmental attributes may be determined. The determined balancing units may be compared to balancing units stored in balancing account(s) for the respective environmental attribute(s).

If the balance of the respective balancing account(s) for the respective environmental attribute(s) is not sufficient, the environmental attribute(s) is rejected. If the balancing units are available, the balancing units are deducted from the respective balancing account(s) and the environmental attribute(s) are assigned to the chemical product.

Assigning or attributing the one or more environmental attribute(s) to the chemical product identifier may include generating a digital asset that includes the chemical product identifier and the one or environmental attribute(s) of at least one of the first input material and the second input material and linking the digital asset to the chemical product. For example, referring to FIG. 2, operating system 120 may generate a digital asset that includes the chemical product identifier and the one or environmental attribute(s) of one or more input material(s).

The present disclosure comprises the step (a1) providing multiple input material(s) associated with one or more environmental attribute(s) to the chemical production network, including a first input material and a second input material. Referring to FIG. 2, Input materials 202-206 are provided to chemical production network 110 at the feed-in point 212. The input materials may include conventional fossil feedstock 202 (e.g., naphtha) as well as sustainable input materials 204-206. The sustainable input materials 204-206 may include renewable input materials (such as biogas and/or bio-naphtha) and/or recycled input materials (e.g., pyrolysis oil). After they are delivered to chemical production network 110, the conventional input materials 202 and the sustainable input materials 204-206 may be combined (e.g., by being fed into the same tank) as they enter the chemical production process.

The present disclosure comprises the step (b1) providing a first input material data associated with the first input material and a second input material data associated with the second input material.

Referring to FIG. 2, input material data for sustainable input material 204 is provided to operating system 120 at 222. Similarly, input material data for sustainable input material 206 is provided to operating system 120 at 224. For example, the goods receipt (and/or a BOM and/or a chemical production recipe) including the input material data for each of the sustainable input materials may be electronically provided to operating system 120 when sustainable materials 204-206 are delivered to chemical production network 110. Operating system 120 may receive input material data 222-224 through an interface to a local or a remote database or an ERP system, in particular its supply chain module, or any computing system or apparatus, such as a centralized or decentralized computing system or apparatus including processing and storage. The input material data for each input material may hence be gathered from an ERP system or any computing system or apparatus, such as a centralized or decentralized computing system or apparatus including processing and storage. In some cases, the input material data of each input material is gathered through an interface to more than one database. It therefore may be necessary to convert the information retrieved from different databases into a single format to allow further processing. In particular, the input material data obtained from databases may be attributed to the input material via the identification of an input material in the database that has to be translated to the identification of the input material of the process data used in the process according to the present disclosure

The present disclosure comprises the step (c1) providing at least one balancing account associated with the one or more environmental attribute(s) of the first input material and the second input material, wherein the virtual balancing account includes at least one attribution rule for attributing the environmental attributes associated with the input material to a chemical product. Referring again to FIG. 2, operating system 120 may credit a virtual balancing account (which may also be referred to as digital inventory) 232 with the amount of conventional feedstock that was created by a virtual production process(es). Operating system 120 may also convert the environmental attributes to balancing units and allocate or credit those balancing units to digital BU inventories (or virtual balancing accounts) 234-236. As described above with reference to FIG. 8b and FIGS. 15a-c, balancing accounts may include attribution rule(s) (also called allocation rules) to assign (or attribute) environmental attributes to a chemical product.

The present disclosure comprises the step (d1) providing a chemical product identifier associated with the chemical product and at least one target environmental attribute. Referring to FIGS. 1-2, operating system 120 may provide a chemical product identifier for one or more of chemical products 262-264. The chemical product identifier may include one or more identifier(s) relating to the chemical product. The identifier may relate to a chemical product class, a specific chemical product and/or properties of the chemical product such as environmental properties. The identifier may include a unique number uniquely associated with the chemical product class, the specific chemical product and/or the properties of the chemical product. The identifier may include one or more specific identifier(s), such as chemical product class identifier, specific chemical product identifier and/or property of the chemical product identifier. Such specific identifier(s) may be uniquely linked to the chemical product. For example, one or more property identifier(s) may be uniquely linked to the chemical product identifier. The chemical product identifier may be uniquely linked to the specific chemical product. This way the chemical product can be uniquely linked to a digital twin of the chemical product specifying specific properties of the chemical product.

The present disclosure comprises the step (e1) based on the chemical product identifier and the target environmental attribute, selecting at least one attribution rule and (f1) determining via the at least one attribution rule at least one account for attributing one or more environmental attribute(s) from the at least one account to the chemical product identifier. Referring to FIGS. 1-2, operating system 120 may select at least one attribution rule based, at least in part, on the chemical product identifier and the target environmental attribute. The attribution rule may map the environmental attribute type to the chemical product type. For example, the attribution rule may include metadata related to the environmental attribute type and to the chemical product type. Similarly, the input material account may include metadata related to the environmental attribute type and to the chemical product type. Based on the matching of metadata the input material account may be selected. This way the account for attributing environmental attributes from account to the chemical product identifier may be determined The present disclosure comprises the step (g1) assigning or attributing the one or more environmental attribute(s) to the chemical product identifier. As for example illustrated in FIG. 1, the chemical production network may be operated by the operating system configured to register inbound environmental attributes, to assign outbound environmental attributes and/or to manage inbound registration as well as outbound assignment via allocation rules. The allocation rules may include allocation schemes as illustrated in FIGS. 6a-c. The chemical production network may include the network as described in the context of FIG. 5 or 7.

The chemical product may be produced from input material that entered the chemical production network. Chemical product data including the chemical product identifier may be provided. The chemical product data may further include input material data associated with the input materials used to produce the chemical product(s), process data associated with the production chain for producing the chemical products and/or data related to the chemical product data, such as a chemical product specification or a chemical product amount.

Based on the input material data environmental attributes associated with the input material(s) used to produce the chemical product(s) may be determined. The input material data may specify the total amount of input material(s) that entered the production chain for producing the amount of chemical product. The input material data may further specify the environmental attributes available for the respective input material(s). From the total amount of input material(s), the number of balancing units for respective environmental attributes available for such input material(s) may be determined. This way the maximum number of balancing units for the respective environmental attribute attributable to the chemical product may be determined.

Based on the environmental attribute(s) attributable to the chemical product, the number of balancing units corresponding to the determined environmental attributes may be determined. The determined balancing units may be compared to balancing units stored in balancing account(s) for the respective environmental attribute(s).

If the balance of the respective balancing account(s) for the respective environmental attribute(s) is not sufficient, the environmental attribute(s) is rejected. If the balancing units are available, the balancing units are deducted from the respective balancing account(s) and the environmental attribute(s) are assigned to the chemical product.

Assigning or attributing the one or more environmental attribute(s) to the chemical product identifier may include generating a digital asset that includes the chemical product identifier and the one or environmental attribute(s) of at least one of the first input material and the second input material and linking the digital asset to the chemical product. For example, referring to FIG. 2, operating system 120 may generate a digital asset that includes the chemical product identifier and the one or environmental attribute(s) of one or more input material(s).

The present disclosure comprises the step (a2) providing input material data associated with the input material to an operating system of the chemical production network. As described above with reference to FIGS. 1-2 and 8a, on entry of the input material, the corresponding input material data may be provided via a network to the interface of the operating system. A data provider may be configured to provide material data related to the one or more input material(s) and respective environmental attributes to a computing interface configured to allocate the environmental attributes. The material data may be provided on, prior to or after providing the one or more input material(s) at entry points to the chemical production network. The input material identifier may be associated with the physical entity of the input material entering the chemical production network.

The present disclosure comprises the step (b2) producing environmental attributes associated with the input material via a virtual production process. Referring to FIG. 2, operating system 120 may initiate a virtual production step after it receives the input material data for sustainable materials 204-206. Virtual production refers to receiving input material data for a sustainable input material and producing environmental attributes (based on the sustainable input material) and also “producing” conventional input material data (e.g., data describing the corresponding amount and/or value of the conventional input material).

For example, with reference to FIGS. 2 and 3, operating system 120 initiates virtual production process 300 when it receives input material data for sustainable input material(s) (e.g., 222-224). Using input material data 222-224, virtual production process 300 may parse the input material data and apply a corresponding recipe. For example, virtual production process 300 may determine the volume (or mass) and type of sustainable input material that was received from the input material data. It may then apply virtual production step(s) 320 to the sustainable input material 310. Virtual production step(s) 320 may “produce” both environmental attributes 330 and conventional input material 340. The amount of conventional input material 340 (virtually) produced may be equal to the amount of sustainable input material 310.

Referring again to FIG. 2, after the virtual production process, operating system 120 may credit digital inventory (which may also be referred to as a virtual balancing account) 232 with the amount of conventional feedstock that was created by the virtual production process(es). Operating system 120 may also convert the environmental attributes to balancing units and allocate or credit those balancing units to digital BU inventories (or virtual balancing accounts) 234-236. The conversion may include a conversion factor that takes account of the chemical difference between fossil-based input materials, such as naphtha and methane, and non-fossil input materials, such as pyrolysis oil. The conversion factor may relate to the lower heating value of the pyrolysis oil in relation to the lower heating value of naphtha or methane. The conversion factor may include the ratio of the lower heating value of pyrolysis oil to naphtha or methane. This way the chemical difference between the fossil and the renewable input material can be taken into account.

Digital inventories 234-236 may determine and track both the amount (e.g., volume and/or mass) and the value of sustainable input material 204-206, respectively. For example, operating system 120 may parse input material data 222 to determine the amount of sustainable input materials 204 that was received. Similarly, operating system 120 may parse input material data 224 to determine the amount of sustainable input material 206 that was received. Operating system 120 may then credit digital inventories 234 and 236, respectively, with the amount of sustainable input materials that were received.

Operating system 120 may also determine a value associated with the balancing units it credits to digital inventories. For example, operating system 120 may compute the difference in cost between sustainable input materials 204-206 and corresponding equivalent fossil input materials to determine the value of the balancing units. Operating system 120 may use average price, actual price, market price or other suitable values to determine the cost of the equivalent amount of fossil input materials. Operating system 120 stores and tracks the amounts and values corresponding to sustainable input materials in digital inventories 234-236. For example, the balancing units stored in digital inventories 234-236 may include the amount and/or value information corresponding to sustainable inputs 204-206.

The present disclosure comprises the step (c2) allocating the environmental attributes associated with the input material to a virtual balancing account, wherein the virtual balancing account includes at least one attribution rule for attributing the environmental attributes associated with the input material to a chemical product. Referring again to FIG. 2, operating system 120 may credit a virtual balancing account (which may also be referred to as digital inventory) 232 with the amount of conventional feedstock that was created by a virtual production process(es). Operating system 120 may also convert the environmental attributes to balancing units and allocate or credit those balancing units to digital BU inventories (or virtual balancing accounts) 234-236. As described above with reference to FIG. 8b and FIGS. 15a-c, balancing accounts may include attribution rule(s) (also called allocation rules) to assign (or attribute) environmental attributes to a chemical product.

The present disclosure comprises the step (d2) providing a chemical product identifier associated with the chemical product and at least one target environmental attribute. Referring to FIGS. 1-2, operating system 120 may provide a chemical product identifier for one or more of chemical products 262-264. The chemical product identifier may include one or more identifier(s) relating to the chemical product. The identifier may relate to a chemical product class, a specific chemical product and/or properties of the chemical product such as environmental properties. The identifier may include a unique number uniquely associated with the chemical product class, the specific chemical product and/or the properties of the chemical product. The identifier may include one or more specific identifier(s), such as chemical product class identifier, specific chemical product identifier and/or property of the chemical product identifier. Such specific identifier(s) may be uniquely linked to the chemical product. For example, one or more property identifier(s) may be uniquely linked to the chemical product identifier. The chemical product identifier may be uniquely linked to the specific chemical product. This way the chemical product can be uniquely linked to a digital twin of the chemical product specifying specific properties of the chemical product.

The present disclosure comprises the step (e2) selecting at least one attribution rule based, at least in part, on the chemical product identifier and the target environmental attribute and step (f2) determining via the at least one attribution rule at least one account for attributing one or more environmental attribute(s) from the at least one account to a digital asset the chemical product identifier. Referring to FIGS. 1-2, operating system 120 may select at least one attribution rule based, at least in part, on the chemical product identifier and the target environmental attribute. The attribution rule may map the environmental attribute type to the chemical product type. For example, the attribution rule may include metadata related to the environmental attribute type and to the chemical product type. Similarly, the input material account may include metadata related to the environmental attribute type and to the chemical product type. Based on the matching of metadata the input material account may be selected. This way the account for attributing environmental attributes from account to the chemical product identifier may be determined.

The present disclosure comprises the step (g2) generating a digital asset that includes the chemical product identifier and the one or environmental attributes. Referring to FIG. 2, operating system 120 may generate a digital asset such as an identifier (e.g., including a chemical product identifier and environmental attribute identifiers) as described in the context of FIGS. 14a and 14b. The digital asset may include the chemical product identifier. The digital asset may include one or more environmental attribute(s) such as the product carbon footprint, recycled content or bio-based content. The digital asset may relate to one or more environmental attribute(s) such as the product carbon footprint, recycled content or bio-based content. The digital asset may include a digital representation of one or more environmental attribute(s) such as the product carbon footprint, recycled content or bio-based content.

The digital asset may further include or relate to authentication and/or authorization information linked to the chemical product identifier. The authentication and/or authorization information may be provided for authentication and/or authorization of a data providing service 2208 and/or data consuming service 2210. The chemical product identifier may include or relate to a decentral identifier, that is uniquely associated with the chemical product. The decentral identifier may be connected to the digital representation of the environmental attributes. The digital representation may include a representation for accessing the environmental attributes or parts thereof. The decentral identifier may include a Universally Unique IDentifier (UUID) or a Digital IDentifier (DID). The decentral identifier may include any unique identifier uniquely associated with a data owner and/or chemical product. The data owner may be the producer of the chemical product. Via the decentral identifier and its unique association with the data owner and/or chemical product, access to the material configuration data may be controlled by the data owner.

The digital asset including the digital representation of one or more environmental attribute(s) such as the product carbon footprint, recycled content or bio-based content may be stored in a decentral data base 2200. The one or more environmental attribute(s) such as the product carbon footprint, recycled content or bio-based content may be stored in a data base 2202 associated with the data owner, such as the producer of the chemical product 272.

The present disclosure comprises the step (h2) linking the digital asset to the chemical product. Referring to FIG. 2, operating system 120 may link the digital asset (e.g., 272-274) to a chemical product (e.g., 262-264). Such linking may include a physical or virtual link of identifiers uniquely associated with the physical input material(s) (e.g., 204-206). For physical linking a tag or code may be physically connected to the material, e.g., by printing a QR code on the packaging. For virtual linking different identifiers associated with the physical material may be linked. For example, an order number, a batch number, LOT number or a combination thereof may be linked. The material data may include at least one environmental attribute associated with the respective input material and an amount of input material provided to the entry point of the chemical production network.

The present disclosure has been described in conjunction with preferred embodiments and examples as well. However, other variations can be understood and effected by those persons skilled in the art and practicing the claimed invention, from the studies of the drawings, this disclosure and the claims.

Any steps presented herein can be performed in any order. The methods disclosed herein are not limited to a specific order of these steps. It is also not required that the different steps are performed at a certain place or in a certain computing node of a distributed system, i.e. each of the steps may be performed at different computing nodes using different equipment/data processing.

As used herein “determining” also includes “initiating or causing to determine”, “generating” also includes “initiating and/or causing to generate” and “providing” also includes “initiating or causing to determine, generate, select, send and/or receive”. “Initiating or causing to perform an action” includes any processing signal that triggers a computing node or device to perform the respective action.

In the claims as well as in the description the word “comprising” does not exclude other elements or steps and the indefinite article “a” or “an” does not exclude a plurality. A single element or other unit may fulfill the functions of several entities or items recited in the claims. The mere fact that certain measures are recited in the mutual different dependent claims does not indicate that a combination of these measures cannot be used in an advantageous implementation.

Any disclosure and embodiments described herein relate to the methods, the systems, devices, the computer program element lined out above and vice versa. Advantageously, the benefits provided by any of the embodiments and examples equally apply to all other embodiments and examples and vice versa.

All terms and definitions used herein are understood broadly and have their general meaning.

Any disclosure and embodiments described herein are mere examples for implementing the method, the system or application device disclosed herein and shall not be considered limiting.

1. A computer-implemented method for attributing at least one environmental attribute associated with an input material to one or more chemical products(s), wherein the one or more chemical products(s) are produced by a chemical production network using the input material(s), wherein the chemical production network chemically converts input materials via chemical intermediates to chemical products that exit the chemical production network, the method comprising:
providing input material data associated with the input material to an operating system of the chemical production network;
determining environmental attributes associated with the input material via a virtual production process;
allocating the environmental attributes associated with the input material to a virtual balancing account, wherein the virtual balancing account includes at least one attribution rule for attributing the environmental attributes associated with the input material to a chemical product;
providing a chemical product identifier associated with the chemical product and at least one target environmental attribute;
based on the chemical product identifier and the target environmental attribute, selecting at least one attribution rule;
determining via the at least one attribution rule at least one account for attributing one or more environmental attribute(s) from the at least one account to the chemical product identifier; and
assigning or attributing the one or more environmental attribute(s) to the chemical product identifier.

2. The method of claim 1, wherein determining the environmental attributes associated with the input material via the virtual production process further comprises determining the amount of the input material.

3. The method of claim 1 or 2, wherein determining the environmental attributes associated with the input material via the virtual production process further comprises determining a value associated with the input material.

4. The method of claim 3, wherein the value associated with the input material is related to a difference in cost between the input material and a corresponding amount of fossil input material.

5. The method of claim 4, wherein the difference in cost between the input material and a corresponding amount of fossil input material is an average difference in cost between the input material and a corresponding amount of fossil input material.

6. The method of claim 1, wherein assigning or attributing the one or more environmental attribute(s) to the chemical product identifier comprises producing a digital asset that specifies a chemical product with the combination of the chemical product identifier and the one or more environmental attributes.

7. The method of claim 6, wherein the digital asset uniquely specifies a chemical product with the combination of the chemical product identifier and the one or more environmental attributes.

8. The method of claim 7, wherein the chemical product identifier is associated with a product specification for the chemical product.

9. The method of claim 6, wherein the digital asset includes a value associated with the input material, wherein the value associated with the input material is related to a difference in cost between the input material and a corresponding amount of fossil input material.

10. The method of claim 1, further comprising providing multiple input material(s) associated with one or more environmental attribute(s) to the chemical production network, including a first input material and a second input material.

11. The method of claim 10, wherein providing input material data associated with the input material further comprises:
providing a first input material data associated with the first input material; and
providing a second input material data associated with the second input material.

12. The method of claim 11, further comprising:
providing at least one balancing account associated with one or more environmental attribute(s) of the first input material and the second input material.

13. The method of claim 12, wherein assigning or attributing the one or more environmental attribute(s) to the chemical product identifier comprises:
generating a digital asset that includes the chemical product identifier and the one or environmental attribute(s) of at least one of the first input material and the second input material; and
linking the digital asset to the chemical product.

14. An apparatus for assigning or attributing at least one environmental attribute associated with input material(s) to one or more chemical product(s), wherein the one or more chemical product(s) are produced by a chemical production network using at least one of the input material(s), the apparatus comprising:
a virtual production module configured to
receive input material data associated with the at least one input material and to
determine environmental attributes associated with the at least one input material;
a balancing module configured to provide at least one account for balancing the environmental attributes produced by the virtual production module;
an attribution module configured to provide at least one attribution rule for attributing environmental attributes associated with input material(s) to chemical product(s);
a digital asset provider configured to provide at least one chemical product identifier associated with the chemical product and at least one target environmental attribute for the chemical product; and
an outbound allocator configured to
select based on the chemical product identifier and the target environmental attribute at least one attribution rule,
determine at least one account for assigning or attributing one or more environmental attribute(s) from the account to the digital asset via the at least one attribution rule, and
assigning or attributing the one or more environmental attribute(s) to the digital asset.

15. The apparatus of claim 14, wherein the virtual production module is further configured to
determine an amount of the input material; and
determine a value associated with the input material.
